(19) United States
(12) Patent Application Publication
(10) Pub. No.: US 2026/0265729 A1
(43) Pub. Date: Sep. 10, 2026

(54) Generation of adeno-associated virus capsid libraries for insect cells

(71) Applicant: uniQure biopharma B.V., Amsterdam (NL)

(72) Inventors: - Anggakusuma, Amsterdam (NL); Sebastiaan Menno Bosma, Amsterdam (NL); Elena Maria Santidrian-Yebra-Pimentel, Amsterdam (NL)

(73) Assignee: uniQure biopharma B.V., Amsterdam (NL)

(21) Appl. No.: 19/473,493

(22) PCT Filed: Apr. 18, 2024
(86) PCT No.: PCT/EP2024/060492

§ 371 Date: Oct. 8, 2025

(30) Foreign Application Priority Data
Apr. 18, 2023 (EP) 23168403.6

(51) Int. Cl. C12N 15/10; C07K 14/005; C12N 15/86

(52) U.S. Cl. CPC C12N 15/1065; C07K 14/005; C12N 15/86; C12N 2710/14043; C12N 2750/14122; C12N 2750/14143; C12N 2830/008; C12N 2830/60

(57) ABSTRACT

The present invention relates to means and methods for generating adeno-associated virus capsid libraries using insect host cells. In particular, the invention relates to novel DNA constructs and methods using these constructs for generating adeno-associated virus capsid libraries in insect host cells that minimize the occurrence of cross-packaging and mosaicism in the libraries generated. The invention further relates to method for identifying AAV capsid variants with one or more desired characteristics, such as e.g. the combination of CNS tropism and detargeting of peripheral organs.

### FIELD OF THE INVENTION

The present invention relates to the fields of medicine, molecular biology, gene therapy and insect cell culture. In particular, the invention relates to means and methods for generating adeno-associated virus capsid libraries for insect cells.

### BACKGROUND OF THE INVENTION

Adeno-associated virus (AAV) has emerged as a preferred platform for gene therapy. Yet, the success of AAV-based therapeutic strategies has been limited by pre-existing immunity, nonspecific tropism and inefficient production at large scale. To address these challenges, efforts have been made in the engineering of novel AAV capsids that are capable of evading neutralizing antibodies (NAbs) and/or that have improved tissue tropism, or an altered specificity thereof.

AAV capsid libraries have been widely and successfully applied to select capsids with improved traits, e.g. in directed evolutionary studies. Production of such AAV libraries leverages the mammalian production platform in Hek293 cells. Libraries produced on this platform have sufficient depth and titer to perform these type of studies. In this approach, AAV libraries are generated by transfection of highly diverse libraries of plasmids encoding capsid mutants expressed in the “natural” AAV genome context, where the two viral genes (Rep and Cap) are flanked by inverted terminal repeats. AAV variants with desirable properties can then be isolated by successive rounds of phenotypic selection and recovery of the viral DNA. This method, therefore, critically depends on an excellent genome—capsid “identity” correlation. However, production of AAV capsid libraries in the Hek293 platform requires the transfection of high quantities of plasmid DNA, making them prone to transgene cross-packaging and mosaicism, in which particles are comprised of genomes and capsid monomers derived from different library members (see e.g. Schmit et al., 2019, Mol Ther Methods Clin Dev, 17:107-121).

Furthermore, capsids selected from libraries produced in the mammalian platform may not be amenable to efficient production in an insect cell-based platform.

There is therefore still a need in the art to address the above short-comings and to provide for means and methods for generating AAV libraries in insect cells that minimize the occurrence of cross-packaging and mosaicism in the libraries generated. It is an object of the invention to provide for such means and methods.

### SUMMARY OF THE INVENTION

In a first aspect, there is provided a nucleic acid construct comprising: i) a nucleotide sequence encoding an adeno-associated virus (AAV) capsid variant, wherein the nucleotide sequence encodes an mRNA, translation of which in an insect cell produces the AAV capsid variant, and wherein the nucleotide sequence is operably linked to a promoter for driving expression in an insect cell; ii) an enhancer element that is operably linked to the promoter, wherein the enhancer element is dependent on a transcriptional transregulator, and wherein introduction of the transcriptional transregulator into an insect cell comprising the nucleic acid construct, induces transcription from the promoter; iii) at least one AAV inverted terminal repeat (ITR) sequence; iv) optionally, a reporter gene operably linked to a promoter for driving expression in a mammalian cell; and, v) optionally, a barcode.

In one embodiment of the nucleic acid construct, the transcriptional transregulator is a baculoviral immediate-early protein (IE1) or its splice variant (IE0) and the transcriptional transregulator-dependent enhancer element is a baculoviral homologous region (hr) enhancer element, wherein preferably the baculovirus is Autographa californica multicapsid nucleopolyhedrovirus. In one embodiment, the hr enhancer element comprises at least one copy of the hr 28-mer sequence CTTTACGAGTAGAATTCTACGCGTAAAA and/or at least one copy of a of a sequence of which at least 20, 21, 22, 23, 24, 25, 26, or 27 nucleotides are identical to sequence CTTTACGAGTAGAATTCTACGCGTAAAA and which binds to a baculoviral IE1 protein, and wherein the hr enhancer element, when operably linked to an expression cassette comprising a reporter gene operably linked to the polH promoter, a) under non-inducing conditions, the expression cassette with the hr enhancer element produces less reporter transcript than an otherwise identical expression cassette which comprises the hr2-0.9 element, or the cassette with the hr enhancer element produces less than a factor 1.1, 1.2, 1.5, 2, 5 or 10 of the amount reporter transcript produced by an otherwise identical expression cassette which comprises the hr4b element; and, b) under inducing conditions, the expression cassette with the hr enhancer element produces at least 50, 60, 70, 80, 90 or 100% of the amount of reporter transcript produced by an otherwise identical expression cassette which comprises the hr4b or the hr2-0.9 element, wherein more preferably the hr enhancer element is selected from the group consisting of hr1, hr2-0.9, hr3, hr4b and hr5, of which hr4b and hr5 are preferred, of which hr4b is most preferred.

In one embodiment of the nucleic acid construct, the promoter for driving expression in an insect cell is a baculoviral promoter, preferably, a late or very late baculoviral promoter, more preferably, a promoter selected from the group consisting of the polH, p10, p6.9 and pSel120 promoters.

In one embodiment, the nucleic acid construct further comprises a recognition sequence for an endonuclease, preferably a recognition sequence for a site-specific endonuclease. In one embodiment, the recognition sequence is a recognition sequence for a protelomerase, preferably a phage N15 TeIN protelomerase.

In one embodiment, of the nucleic acid construct, the recognition sequence for the endonuclease is not flanked by at least one AAV ITR sequence, preferably is not present in between the two AAV ITR sequences, such that the recognition sequence is not incorporated into the genome of a rAAV vector produced in the insect cell.

In one embodiment, the nucleic acid construct comprises in a 5′ to 3′ order: a) a first AAV ITR sequence; b) the enhancer element; c) an expression cassette comprising the nucleotide sequence encoding the AAV capsid variant, operably linked to the promoter for driving expression in the insect cell; and, d) a second AAV ITR, wherein the recognition sequence is present at at least one of i) upstream of the first AAV ITR and ii) downstream of the second AAV ITR, wherein optionally, the reporter gene operably linked to the promoter for driving expression in a mammalian cell is present between a) and b), b) and c) or c) and d), and wherein optionally, a barcode is present between a) and b), b) and c) or c) and d) and wherein, preferably the recognition sequence is a unique recognition sequence.

In one embodiment of the nucleic acid construct, at least one of: a) the promoter for driving expression in a mammalian cell is a cell type and/or tissue specific promoter, preferably a promoter selected from the group consisting of human synapsin promoter (hSynl), transthyretin promoter (TTR), cytokeratin 18, cytokeratin 19, unc-45 myosin chaperon B (unc45b) promoter, cardiac troponin T (cTnT) promoter, glial fibrillary acidic protein (GFAP) promoter, myelin basic protein (MBP) promoter, and methyl-CpG-binding protein 2 (Mecp2) promoter; b) the reporter gene encodes a colorimetric, fluorescent or bioluminescent reporter protein, preferably a reporter protein is selected from the group consisting of: secreted alkaline phosphatase (SEAP), β-galactosidase EGFP, mCherry, mCloverS, mRubyS, mApple, iRFP, tdTomato, mVenus, YFP, RFP, firefly luciferase, sea pansy luciferase and nanoluciferase; and, c) the barcode is a nucleotide sequence of 4-18 nucleotides long.

In a second aspect, there is provided a nucleic acid molecule comprising a nucleic acid construct as described herein. In one embodiment, the nucleic acid molecule is a linear nucleic acid molecule.

In one embodiment, the linear nucleic acid molecule is obtainable by digestion with a site specific endonuclease that cuts at the recognition sequence in the nucleic acid construct.

In one embodiment, the linear nucleic acid molecule is a nucleic acid molecule wherein at least one end of the linear nucleic acid molecule is protected against exonuclease degradation, preferably both ends of the linear nucleic acid molecule are protected against exonuclease degradation.

In one embodiment of the linear nucleic acid molecule, the endonuclease is a protelomerase, preferably a phage N15 TeIN protelomerase, and wherein at least one end of the linear nucleic acid molecule is covalently closed, or wherein preferably both ends of the linear nucleic acid molecule are covalently closed.

In one embodiment of the linear nucleic acid molecule, the locations of elements i) to v) and the recognition sequence in the construct are such that expression of AAV Rep proteins in an insect cell comprising the nucleic acid construct, effects replication and packaging into AAV capsids of a DNA fragment comprising elements i) to v).

In a third aspect, there is provided a library comprising a plurality of nucleic acid molecules comprising a nucleic acid construct as described herein.

In one embodiment, the library is a library wherein, in each member of the library, the nucleotide sequence encoding the AAV capsid variant differs by at least one nucleotide from nucleotide sequences encoding the AAV capsid variants in other members in the library, and wherein preferably, in each member of the library, the nucleotide sequence encoding the AAV capsid variant, encodes a capsid variant that differs by at least one amino acid from AAV capsid variants encoded by other members in the library.

In a fourth aspect, there is provided an insect cell comprising a nucleic acid molecule comprising a nucleic acid construct as described herein or comprising a library comprising (plurality) of such nucleic acid molecules comprising a nucleic acid construct as described herein.

In one embodiment, the insect cell further comprises a baculoviral vector comprising at least one expression cassette for expression of AAV Rep proteins in the insect cell.

In a fifth aspect, there is provided a library comprising a plurality of AAV capsid variants, wherein members in the library comprise a nucleic acid that is replicated and packaged from a member in the library of nucleic acid molecules comprising a nucleic acid construct as described herein, e.g. as described in the third aspect.

In a sixth aspect, there is provided a method for preparing a library of AAV capsid variants. In one embodiment, the method for preparing a library of AAV capsid variants comprises the steps of: a) providing a library comprising a plurality of nucleic acid construct as defined herein above, wherein the amino acid sequence of an AAV capsid variant of each member in the library as encoded in the construct of that member, differs by at least one amino acid from amino acid sequences of AAV capsid variants encoded in constructs of other members in the library; b) amplifying the library of the nucleic acid constructs obtained in a), preferably by rolling circle amplification; c) digestion of the amplified library of the nucleic acid constructs obtained in b) by a site-specific endonuclease that cuts at a recognition sequence in the nucleic acid construct to produce a library comprising linearised amplified nucleic acid constructs of unit length; d) transfection of the library comprising linearised amplified nucleic acid constructs obtained in c) into insect cells; e) infection of the insect cells with a baculoviral vector comprising at least one expression cassette for expression of AAV Rep proteins in the insect cells and culturing of the cells; and, f) recovery of a library of AAV capsid variants produced by the insect cells in e).

In one embodiment, the method for preparing a library of AAV capsid variants is a method wherein the library in a) is provided by: i) providing a nucleic acid construct as defined in the first aspect; and, ii) replacing a fragment comprising at least a part of the nucleotide sequence encoding the AAV capsid variant in the construct, by a library of fragments, wherein the amino acid sequence of an AAV capsid variant of each member in the library as encoded in the fragment of that member, differs by at least one amino acid from amino acid sequences of AAV capsid variants encoded in constructs of other members in the library.

In one embodiment, the method for preparing a library of AAV capsid variants is a method wherein—in step ii), the replacement is performed by Gibson assembly;—in step b), the rolling circle amplification is performed by using a φ29 DNA polymerase, preferably, in combination with random primers; and/or—in c), the ends of the linearised amplified library of the nucleic acid constructs of unit length are provided with protection against exonuclease degradation. In one embodiment,—in step c), the endonuclease is a phage N15 TeIN protelomerase, providing covalently closed ends to the linearised amplified library of the nucleic acid constructs of unit length.

In a seventh aspect, there is provided a method for identifying an AAV capsid variant with a desired characteristic. In one embodiment, the method comprises: a) providing a library of AAV capsid variants as defined herein above, or as obtained in a method for preparing a library of AAV capsid variants as defined herein above; b) contacting the library with a culture of cells, an organoid or a tissue, or administering the library to a non-human animal; c) allowing AAV capsid variants in the library to transduce the cells, the organoid, the tissue or cells in the animal; and, d) identifying at least one AAV capsid variant that has transduced at least one desired cell-type in the cells, the organoid, the tissue or in the animal in b), as an AAV capsid variant with the desired characteristic, and optionally recovery of the AAV capsid variant with the desired characteristic from the desired cell.

In one embodiment of the method for identifying an AAV capsid variant with a desired characteristic, step d) comprises detection of transduction of at least the desired cell by detecting expression of the reporter gene in the desired cell. In one embodiment of the method the variant with the desired characteristic is identified by sequencing at least one of i) the barcode and ii) a region of the DNA construct encoding the at least one amino acid difference.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer or a neurologic disease, varies depending upon the manner of administration, the age, body weight, and general health of the subject. Ultimately, the attending physician or veterinarian will decide the appropriate amount and dosage regimen. Such amount is referred to as an “effective” amount, which may be determined for instance as genome copies per kilogram (GC/kg) or as GC per dose. Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” and “similarity” also means the degree of sequence relatedness between amino acid or nucleic acid sequences, as the case may be, as determined by the match between strings of such sequences. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, more preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly (A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant parvoviral proteins and/or nucleic acids, such as baculoviral vectors for expression of parvoviral proteins and/or nucleic acids in insect cells.

A “parvoviral vector” is defined as a recombinantly produced parvovirus or parvoviral particle that comprises a polynucleotide to be delivered into a host cell, either in vivo, ex vivo or in vitro. An adeno-associated virus (AAV) vector is an example of a parvoviral vector. Herein, a parvoviral or AAV vector refers to the polynucleotide comprising part of the parvoviral genome, usually at least one ITR, and a transgene, which polynucleotide is preferably packaged in a parvoviral or AAV capsid.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

The term “library” as used herein refers to a collection of elements or members that differ from one another in at least one aspect. For example, a library of nucleic acid molecules or a library of nucleic acid constructs is a collection of at least two nucleic acid molecules or at least two nucleic acid constructs that differ from one another by at least one nucleotide. Likewise, a library of AAV capsid variants is a collection of at least two AAV capsid variants (virions) that differ from one another by at least one nucleotide and/or at least one amino acid in their capsid proteins.

The term “tropism” as used herein refers to the preferential targeting by a virus (e.g., an AAV) of cells of a particular host species or of particular cell types within a host species. For example, a virus that can infect cells of the heart, lung, liver, and muscle has a broader (i.e., increased) tropism relative to a virus that can infect only lung and muscle cells. Tropism can also include the dependence of a virus on particular types of cell surface molecules of the host. For example, some viruses can infect only cells with surface glycosaminoglycans, while other viruses can infect only cells with sialic acid (such dependencies can be tested using various cells lines deficient in particular classes of molecules as potential host cells for viral infection). In some cases, the tropism of a virus describes the virus's relative preferences. For example, a first virus may be able to infect all cell types but is much more successful in infecting those cells with surface glycosaminoglycans. A second virus can be considered to have a similar (or identical) tropism as the first virus if the second virus also prefers the same characteristics (e.g., the second virus is also more successful in infecting those cells with surface glycosaminoglycans), even if the absolute transduction efficiencies are not similar. For example, the second virus might be more efficient than the first virus at infecting every given cell type tested, but if the relative preferences are similar (or 5 identical), the second virus can still be considered to have a similar (or identical) tropism as the first virus. In some embodiments, the tropism of a virion comprising a subject variant AAV capsid protein is not altered relative to a naturally occurring virion. In some embodiments, the tropism of a virion comprising a subject variant AAV capsid protein is expanded (i.e., broadened) relative to a naturally occurring virion. In some embodiments, the tropism of a virion comprising a subject variant AAV capsid protein is reduced relative to a naturally occurring virion.

Any reference to nucleotide or amino acid sequences accessible in public sequence databases herein refers to the version of the sequence entry as available on the filing date of this document.

### DETAILED DESCRIPTION OF THE INVENTION

The present inventors have set out to develop means and methods for generating libraries of AAV capsid variants to be produced in insect cells. They have succeeded in developing such methods, which minimize the occurrence of cross-packaging and mosaicism in the libraries generated while maintaining sufficient depth and complexity methods for generating AAV libraries in insect cells that minimize the occurrence of cross-packaging and mosaicism in the libraries.

In a first aspect, there is provided a nucleic acid construct. In one embodiment, the nucleic acid construct comprises: i) a nucleotide sequence encoding an adeno-associated virus (AAV) capsid variant wherein the nucleotide sequence is operably linked to a promoter for driving expression in an insect cell; ii) an enhancer element that is operably linked to the promoter, wherein the enhancer element is dependent on a transcriptional transregulator, and wherein introduction of the transcriptional transregulator into an insect cell comprising the nucleic acid construct, induces transcription from the promoter; and iii) at least one AAV inverted terminal repeat (ITR) sequence.

In one embodiment, a nucleic acid construct as described herein thus comprises an expression cassette for expression of an AAV capsid variant in insect cells. An “AAV capsid variant” is herein understood as an AAV capsid in which at least one of the capsid proteins differs by at least one amino acid from the corresponding wild type AAV capsid protein.

In one embodiment, a nucleic acid construct as described herein comprises an expression cassette comprising a promoter that is active in insect cells, the promoter being operably linked to a nucleotide sequence encoding an mRNA, translation of which in an insect cell produces the AAV capsid variant. The nucleotide sequence in the expression cassette thus encodes an mRNA comprising an open reading frame translation of which in an insect cell produces the AAV capsid proteins that are necessary for assembly of an AAV capsid variant. The nucleotide sequence in the expression cassette thus encodes an mRNA comprising an open reading frame translation of which in an insect cell produces at least the AAV VP1 and VP3 proteins, as AAV VP2 is not strictly necessary for the complete virus particle formation and an efficient infectivity (Warrington et al., 2004, J Virol. 78:6595-609). In a preferred embodiment, the nucleotide sequence in the expression cassette encodes an mRNA comprising an open reading frame translation of which in an insect cell produces all three of the AAV VP1, VP2 and VP3 proteins. In one embodiment, the expression cassette thus encodes an mRNA comprising a single open reading frame that is capable of being translated into at least the AAV VP1 and VP3 capsid proteins by leaky scanning of the VP1 translation initiation codon and inactivation of the VP2 translation initiation codon. In a preferred embodiment, the expression cassette encodes an mRNA comprising a single open reading frame that is capable of being translated into all three AAV VP1, VP2 and VP3 capsid proteins by leaky scanning of the VP1 and VP2 translation initiation codons.

Thus, in one embodiment, the expression cassette for an AAV capsid variant comprises a nucleotide sequence comprising a single open reading frame encoding all three of the parvoviral (AAV) VP1, VP2, and VP3 capsid proteins, wherein the initiation codon for translation of the VP1 capsid protein is a suboptimal initiation codon that is not ATG as e.g. described by Urabe et al., (2002, supra) and in WO2007/046703. A suboptimal initiation codon for the VP1 capsid protein may be as defined above for the Rep78 protein. More preferred suboptimal initiation codons for the VP1 capsid protein may be selected from ACG, TTG, CTG and GTG, of which CTG and GTG are most preferred. In an alternative embodiment, the expression cassette comprises a nucleotide sequence comprising a single open reading frame encoding all three of the parvoviral (AAV) VP1, VP2, and VP3 capsid proteins, wherein the initiation codon for translation of the VP1 capsid protein is ATG and wherein the mRNA coding for the VP1 capsid protein as encoded in the nucleotide sequence comprises an alternative start codon which is out of frame with the open reading frame the VP1 capsid protein (as described in WO2019/016349). Preferably, the alternative start codon is selected from the group consisting of CTG, ATG, ACG, TTG, GTG, CTC and CTT, of which ATG is preferred. Preferably, the AAV capsid proteins are AAV5 serotype capsid proteins. Preferably in this embodiment, the nucleotide sequence comprises an alternative open reading frame starting with the alternative start codon that encompasses said ATG translation initiation codon for VP1, whereby preferably, the alternative open reading frame following the alternative start codon encodes a peptide of up to 20 amino acids.

In one embodiment of the expression cassette for an AAV capsid variant as described herein, the promoter for driving expression in an insect cell is a baculoviral promoter. In one embodiment, the baculoviral promoter is a late or very late baculoviral promoter, such as a promoter is selected from the group consisting of the polH, p10, p6.9 and pSel120 promoters. In a preferred embodiment, the late or very late baculoviral promoter is the baculoviral p10 promoter.

In one embodiment of the expression cassette for an AAV capsid variant as described herein, the expression cassette comprises a polyadenylation signal, preferably polyadenylation signal that effects polyadenylation of the mRNA expressed from the expression cassette in an insect cell.

In one embodiment, a nucleic acid construct as described herein thus further comprises an enhancer element that is operably linked to the promoter (for driving expression of the AAV cap variant in an insect cell). An “enhancer element” or “enhancer” is meant to define a sequence which enhances the activity of a promoter (i.e. increases the rate of transcription of a sequence downstream of the promoter) which, as opposed to a promoter, does not possess promoter activity, and which can usually function irrespective of its location with respect to the promoter (i.e. upstream, or downstream of the promoter). Enhancer elements are well-known in the art. Non-limiting examples of enhancer elements (or parts thereof) which could be used in the present invention include baculovirus enhancers and enhancer elements found in insect cells. It is preferred that the enhancer element increases in a cell the mRNA expression of a gene, to which the promoter it is operably linked, by at least 25%, more preferably at least 50%, even more preferably at least 100%, and most preferably at least 200% as compared to the mRNA expression of the gene in the absence of the enhancer element. mRNA expression may be determined for example by quantitative RT-PCR.

Herein it is preferred to use an enhancer element to enhance the expression of the AAV capsid variant. In one embodiment the at least one enhancer element that is operably linked to the promoter in the expression cassette for an AAV capsid variant as defined herein, is an enhancer element that is dependent on a transcriptional transregulator. A transcriptional transregulator-dependent enhancer element is herein understood as an enhancer element that activates transcription of a promoter operably linked thereto when bound by a transcriptional transregulator protein provided in trans. In the absence of transcriptional transregulator protein there will thus be little or no transcription by the promoter operably linked to the enhancer.

Thus, in one embodiment, the transcriptional transregulator-dependent enhancer element comprises at least one baculovirus enhancer element and/or at least one ecdysone responsive element. Preferably the transcriptional transregulator is a baculoviral immediate-early protein (IE1) or its splice variant (IE0) and the transcriptional transregulator-dependent enhancer element is a baculoviral homologous region (hr) enhancer element, wherein preferably the baculovirus is Autographa californica multicapsid nucleopolyhedronvirus. IE1 is a highly conserved, 67-kDa DNA binding protein that transactivates baculovirus early gene promoters and supports late gene expression in plasmid transfection assays (see e.g. Olson et al., 2002, J Virol., 76:9505-9515). AcMNPV IE1 possesses separable domains that contribute to promoter transactivation and DNA binding. The N-terminal half of this 582-residue phosphoprotein contains transcriptional stimulatory domains from residue 8 to 118 and 168 to 222. IE1 binds to the ~28-bp imperfect palindrome (28-mer) that constitutes repetitive sequences within multiple homologous regions (hrs) found dispersed throughout the AcMNPV genome. The hr 28-mer is the minimal sequence motif required for IE1-mediated enhancer and origin-specific replication functions.

In one embodiment, the hr enhancer element is selected from the group consisting of hr1, hr2-0.9, hr3, hr4b and hr5, of which hr4b and hr5 are preferred, of which hr4b is most preferred. In an alternative embodiment, the hr enhancer element is a variant hr enhancer element, such as e.g.

a non-naturally occurring designed element. The variant hr enhancer element preferably comprises at least one copy of the hr 28-mer sequence CTTTACGAGTAGAATTCTACGCGTAAAA (SEQ ID NO. 1) and/or at least one copy of a of a sequence of which at least 18, 20, 21, 22, 23, 24, 25, 26, or 27 nucleotides are identical to sequence CTTTACGAGTAGAATTCTACGCGTAAAA (SEQ ID NO. 1) and which preferably binds to the baculoviral IE1 protein, more preferably to the AcMNPV IE1 protein. The variant hr enhancer element is further preferably functionally defined in that when the variant element is operably linked to an expression cassette comprising a reporter gene operably linked to the polH promoter, a) under non-inducing conditions, the cassette with the variant element produces less reporter transcript than an otherwise identical expression cassette which comprises the hr2-0.9 element instead of the variant element, or the cassette with the variant element produces less than a factor 1.1, 1.2, 1.5, 2, 5 or 10 of the amount reporter transcript produced by an otherwise identical expression cassette which comprises the hr4b element instead of the variant element; and b) under inducing conditions, the cassette with the variant element produces at least 50, 60, 70, 80, 90 or 100% of the amount of reporter transcript produced by an otherwise identical expression cassette which comprises the hr4b or the hr2-0.9 element instead of the variant element. Non-inducing conditions are understood as condition in which there is no IE1 protein present in the cell wherein the cassettes are tested and inducing conditions are understood to be conditions wherein sufficient IE1 protein is present to obtain maximal reporter expression with the reference cassettes comprising the hr4b or the hr2-0.9 element. Binding of the variant hr enhancer element to the baculoviral IE1 protein can be assayed by using a mobility shift assay as e.g. described by Rodems and Friesen (J Virol. 1995; 69(9):5368-75).

In one embodiment, the enhancer element is non-leaky but relatively weaker hr such as hr4b. In a preferred embodiment, the hr4b enhancer is combined with the p10 promoter.

In one embodiment, a nucleic acid construct as described herein thus further comprises at least one AAV inverted terminal repeat (ITR) sequence.

Herein, “at least one AAV inverted terminal repeat nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e. being a recognition sequence for trans acting replication proteins, such as e.g. Rep 78 (or Rep68), which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus such AAV replicating in a mammalian cell typically has two ITR sequences. It is, however, possible to engineer an ITR so that binding sites on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred ITR is an AAV2 ITR. For safety reasons it may be desirable to construct a recombinant parvoviral (rAAV) vector that is unable to further propagate after initial introduction into a cell in the presence of a second AAV. Such a safety mechanism for limiting undesirable vector propagation in a recipient may be provided by using rAAV with a chimeric ITR as described in US2003148506.

In one embodiment of the nucleic acid construct, the nucleotide sequence comprising the expression cassette for an AAV capsid variant and the enhancer element operably linked thereto, both as defined above, is flanked by at least one AAV ITR sequence. The term “flanked” with respect to a sequence that is flanked by another element(s) herein indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence that is “flanked” by two other elements (e.g. ITRs), indicates that one element is located 5′ to the sequence and the other is located 3′ to the sequence; however, there may be intervening sequences there between. In a preferred embodiment a nucleotide sequence of (i) is flanked on either side by parvoviral inverted terminal repeat nucleotide sequences.

In one embodiment of the nucleic acid construct, the expression cassette for an AAV capsid variant and the enhancer element operably linked thereto, both as defined above, are flanked by at least one AAV ITR sequence.

In one embodiment of the nucleic acid construct, the nucleotide sequence comprising the expression cassette for an AAV capsid variant and the enhancer element operably linked thereto, both as defined above, that is flanked by at least one parvoviral ITR sequence preferably becomes incorporated into the genome of a recombinant parvoviral (rAAV) vector produced in the insect cell. In one embodiment, the nucleotide sequence comprising the expression cassette for an AAV capsid variant and the enhancer element operably linked thereto, is flanked by two AAV ITR nucleotide sequences, whereby the nucleotide sequence comprising the expression cassette for an AAV capsid variant and the enhancer element is located in between the two AAV ITR nucleotide sequences. In one embodiment, the nucleotide sequence comprising the expression cassette for an AAV capsid variant and the enhancer element can be incorporated into the rAAV vector produced in the insect cell if it is located between two regular ITRs, or is located on either side of an ITR engineered with two D regions. Thus, in a preferred embodiment, there is provided a nucleic acid construct comprising two AAV ITR nucleotide sequences and wherein the nucleotide sequence comprising the expression cassette for an AAV capsid variant and the enhancer element operably linked thereto, is located between the two AAV ITR nucleotide sequences.

Typically, the nucleic acid construct, including ITRs and nucleotide sequence comprising the expression cassette for an AAV capsid variant and the enhancer element operably linked thereto, is 5,000 nucleotides (nt) or less in length in order not to exceed the maximum AAV packaging limit of 5.5 kbp. However, other embodiments, wherein oversized nucleic acid constructs are used, e.g. more than 5,000 nt in length, or even more than the maximum AAV packaging limit of 5.5 kbp do still allow the generation of rAAV vectors and are therefore not excluded.

AAV sequences that may be used as described herein for the production of a recombinant AAV virion, i.e. an AAV vector, in insect cells can be derived from the genome of any AAV serotype. Generally, the AAV serotypes have genomic sequences of significant homology at the amino acid and the nucleic acid levels, provide an identical set of genetic functions, and produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71:6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74:8635-47). Any AAV serotype can be used as source of AAV nucleotide sequences for use in the context of the present invention. Preferably the AAV ITR sequences for use in the context of the present invention are derived from AAV1, AAV2, AAV4 and/or AAV7. Likewise, the Rep (Rep78/68 and Rep52/40) coding sequences are preferably derived from AAV1, AAV2, AAV4 and/or AAV7. The sequences coding for the AAV capsid proteins for use herein are described in more detail below.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells. Modified “AAV” sequences also can be used in this context, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8, AAV9, AAV10, AAV11, AAV12 or AAV13 ITR, or Rep can be used in place of wild-type AAV ITR, or Rep sequences.

Although similar to other AAV serotypes in many respects, AAV5 differs from other human and simian AAV serotypes more than other known human and simian serotypes. In view thereof, the production of rAAV5 can differ from production of other serotypes in insect cells. Where methods of the invention are employed to produce rAAV5, it is preferred that one or more constructs comprising, collectively in the case of more than one construct, a nucleotide sequence comprising an AAV5 ITR, a nucleotide sequence comprises an AAV5 Rep coding sequence (i.e. a nucleotide sequence comprises an AAV5 Rep78). Such ITR and Rep sequences can be modified as desired to obtain efficient production of rAAV5 or pseudotyped rAAV5 vectors in insect cells. E.g., the start codon of the Rep sequences can be modified, VP splice sites can be modified or eliminated, and/or the VP1 start codon and nearby nucleotides can be modified to improve the production of rAAV5 vectors in the insect cell.

In a further embodiment, the nucleic acid construct as described herein, optionally comprises a reporter gene operably linked to a promoter for driving expression in a mammalian cell. As understood herein, the reporter gene preferably encodes a protein that, when expressed in a mammalian cell produces a colorimetric, fluorescent, or luminescent signal that will allow cells that contain and express the reporter gene to be identified from surrounding cells that do not contain the reporter gene.

In one embodiment, the reporter gene encodes a protein that produces a colorimetric signal, such as e.g. genes encoding secreted alkaline phosphatase (SEAP) or β-galactosidase.

In one embodiment, the reporter gene encodes a protein that produces a fluorescent signal, preferred examples of which include EGFP, mCherry, mCloverS, mRubyS, mApple, iRFP, tdTomato, mVenus, YFP and RFP.

In one embodiment, the reporter gene encodes a protein that produces a luminescent signal, preferred examples of which include genes encoding Photinus pyralis (firefly) luciferase and Renilla reniformis (sea pansy) luciferase and nanoluciferase.

In one embodiment of the nucleic acid construct as described herein, the promoter operably linked to the reporter gene for driving its expression in a mammalian cell is a constitutive promoter. In other embodiments, the promoter operably linked to the reporter gene is cell type and/or tissue specific promoter. As will be understood by the skilled person, the cell type and/or tissue specificity of the promoter will depend on the desired cell type and/or tissue-tropism of the AAV capsid variant to be obtained. In some embodiments, the cell type and/or tissue specific promoter for driving expression of the reporter gene in a mammalian cell is a promoter selected from the group consisting of human synapsin promoter (hSynl), transthyretin promoter (TTR), cytokeratin 18, cytokeratin 19, unc-45 myosin chaperon B (unc45b) promoter, cardiac troponin T (cTnT) promoter, glial fibrillary acidic protein (GFAP) promoter, myelin basic protein (MBP) promoter, and methyl-CpG-binding protein 2 (Mecp2) promoter. In a preferred embodiment, the cell type and/or tissue specific promoter is human synapsin promoter (hSynl).

In embodiments, wherein the nucleic acid construct as described herein, optionally comprises a reporter gene operably linked to a promoter for driving expression in a mammalian cell, the reporter gene linked to its promoter is flanked by at least one AAV ITR sequence, preferably present in between the two AAV ITR sequences, such that the reporter gene linked to its promoter—together with the nucleotide sequence comprising the expression cassette for an AAV capsid variant and the enhancer element operably linked thereto, and the optional barcode—is incorporated into the genome of a rAAV vector produced in the insect cell.

In a further embodiment, the nucleic acid construct as described herein, optionally comprises a barcode. The barcode preferably is at least one of a sample identifier and an unique molecular identifier (UMI).

A UMI is a substantially unique sequence or barcode, preferably fully unique, that is specific for a nucleic acid molecule, i.e. unique for each nucleic acid molecule or construct used herein. The UMI may have random, pseudo-random or partially random, or non-random nucleotide sequences. A UMI can be used to uniquely identify the originating molecule from which a sequencing read is derived. For example, reads of amplified nucleic acid molecules can be collapsed into a single consensus sequence from each originating nucleic acid molecule. As indicated above, the UMI may be fully or substantially unique. Fully unique is to be understood herein as that every nucleic acid molecule or construct provided herein comprises a unique tag that differs from all the other tags comprised in further nucleic acid molecules/nucleic acid constructs used herein. Substantially unique is to be understood herein in that each nucleic acid molecule or construct provided herein comprises a random UMI, but a low percentage of these nucleic acid molecules/nucleic acid constructs may comprise the same UMI. Preferably, substantially unique molecular identifiers are used in case the chances of tagging the exact same molecule comprising the same sequence with the same UMI is negligible. Preferably, the UMI is fully unique in relation to a specific sequence of the nucleic acid molecule or construct, e.g. fully unique in relation to a specific capsid variant encoded by the specific sequence of the nucleic acid molecule or construct. The UMI preferably has a sufficient length to ensure this uniqueness. An identifier sequence may range in length from about 2 to 100 nucleotide bases or more, and preferably has a length between about 4-18 nucleotide bases, usually between about 8-12 nucleotide bases. Preferably the identifier sequence does not contain two or more consecutive identical bases. Furthermore, there is preferably a difference between individual identifier sequences of at least two, preferably at least three bases.

In embodiments, wherein the nucleic acid construct as described herein, optionally comprises a barcode, the barcode is flanked by at least one AAV ITR sequence, preferably present in between the two AAV ITR sequences, such that the barcode—together with the nucleotide sequence comprising the expression cassette for an AAV capsid variant and the enhancer element operably linked thereto, and the optional reporter gene—is incorporated into the genome of a rAAV vector produced in the insect cell.

In one embodiment, a nucleic acid construct as described herein further comprises a recognition sequence for an endonuclease, preferably a recognition sequence for a site-specific endonuclease. The recognition sequence can thus be a recognition sequence for a restriction endonuclease or for a programmable endonuclease, such as an RNA-guided CRISPR-endonuclease, a zinc finger nuclease, a TAL-effector nuclease or a meganuclease. In a preferred embodiment, the recognition sequence is a recognition sequence for a protelomerase.

A protelomerase is a site-specific endonuclease that cleaves (restricts or digests) a double-stranded nucleic acid molecule at the recognition sequence for the protelomerase, and simultaneously, the protelomerase covalently closes the double stranded ends resulting from the cleavage of the nucleic molecule. A suitable protelomerases for use in the methods described herein is a bacteriophage protelomerase such as a protelomerase selected from the group consisting of: phiHAP-1 from Halomonas aquamarina, PY54 from Yersinia enterolytica, phiKO2 from Klebsiella oxytoca, VP882 from Vibrio sp. and N15 from Escherichia coli (TelN), or variants of any thereof. The protelomerase may have an amino acid sequence and protelomerase recognition sequence as disclosed in WO2010/086626, which is incorporated herein by reference. Thus, in one embodiment, a nucleic acid construct as described herein comprises a recognition sequence for bacteriophage protelomerase as described above. Preferably, the nucleic acid construct comprises a recognition sequence for a TeIN protelomerase. a recognition sequence for a TeIN protelomerase. In one embodiment, the recognition sequence for a TeIN protelomerase comprises or consists of a sequence of at least 90%, 95%, 96%, 97%, 98%, 99% or 100% sequence identity with the sequence:

(SEQ ID NO: 2)
5′-TATCAGCACACAATTGCCCATTATACGCGCGTATAATGGACTATTG
TGTGCTGATA-3′

In one embodiment, the recognition sequence for the endonuclease is unique within nucleic acid construct as described herein, i.e. there is only a single occurrence of the recognition sequence in the nucleic acid construct.

In one embodiment, in the nucleic acid construct as described herein, the recognition sequence for the endonuclease is not flanked by at least one AAV ITR sequence, preferably is not present in between the two AAV ITR sequences, such that the recognition sequence is not incorporated into the genome of a rAAV vector produced in the insect cell.

In one embodiment, a nucleic acid construct as described herein comprises in a 5′ to 3′ order: a) a first AAV ITR sequence; b) the enhancer element; c) an expression cassette comprising the nucleotide sequence encoding the AAV capsid variant, operably linked to the promoter for driving expression in the insect cell; and, d) a second AAV ITR, wherein the (unique) recognition sequence is present at at least one of i) upstream of the first AAV ITR and ii) downstream of the second AAV ITR, wherein optionally, the reporter gene operably linked to the promoter for driving expression in a mammalian cell is present between a) and b), b) and c) or c) and d), and wherein optionally, a barcode is present between a) and b), b) and c) or c) and d).

In one embodiment, a nucleic acid construct as described herein is a DNA construct, preferably a double stranded DNA construct.

In a second aspect, there is provided a nucleic acid molecule comprising a nucleic acid construct as described herein. In one embodiment, the nucleic acid molecule is a DNA molecule, preferably a double stranded or duplex DNA molecule. In one embodiment, the nucleic acid molecule is a circular nucleic acid molecule.

In one embodiment, the nucleic acid molecule as described herein is a linear nucleic acid molecule, preferably a linear double stranded or duplex DNA molecule. In one embodiment, the linear nucleic acid molecule is obtained or obtainable by digestion with an endonuclease that cuts at a recognition sequence in the nucleic acid construct. In one embodiment, the endonuclease is a site-specific endonuclease, whereby the site-specific endonuclease and its recognition sequence are preferably as defined above. It is thus understood that the linear nucleic acid molecule is obtained or obtainable by digestion of a “precursor” molecule comprising the nucleic acid construct with an endonuclease that cuts at a recognition sequence in the acid construct. The precursor molecule can e.g. be a circular molecule or the precursor molecule can be a long concatemeric molecule comprising many copies of the DNA construct, linked in series that is the product of rolling circle amplification (see methods described below).

In one embodiment, the nucleic acid molecule as described herein is a linear nucleic acid molecule, wherein at least one end of the linear nucleic acid molecule is protected against exonuclease degradation, preferably both ends of the linear nucleic acid molecule are protected against exonuclease degradation. An end of the linear nucleic acid molecule can be protected against exonuclease degradation by means and methods know in the art. For example, an end of a linear nucleic acid molecule can be chemically protected or can be protected by ligation of an adapter comprising chemical modifications that protect against exonucleases, such as a backbone comprising phosphorothioates.

In one embodiment, the nucleic acid molecule as described herein is a linear nucleic acid molecule, wherein at least one end, preferably both ends of the linear nucleic acid molecule are protected against exonuclease degradation, by being covalently closed.

A terminus of a double stranded nucleic acid, wherein the 3′-end terminal nucleotide of the respective upper strand is covalently linked to the 5′-end terminal nucleotide of the respective bottom strand, is annotated herein as a “closed end”. Likewise, a terminus of a double stranded nucleic acid, wherein the 5′-end terminal nucleotide of the respective upper strand is covalently linked to the 3′-end terminal nucleotide of the respective bottom strand, is also annotated herein as a “closed end”. A “closed end” is thus understood herein as a terminus of a double stranded nucleic acid wherein said terminal nucleic acids from opposite strands are covalently linked to each other, as opposed to an “open end” which is understood herein as a terminus of a double stranded nucleic acid wherein said terminal nucleic acids from opposite strands are not covalently linked to each other.

In one embodiment, the nucleic acid molecule as described herein is a linear nucleic acid molecule, wherein at least one end, preferably both ends of the linear nucleic acid molecule are protected against exonuclease degradation by being covalently closed, and wherein the covalently closed end(s) is/are is obtained or obtainable by digestion with a protelomerase as defined above, preferably by digestion with a TeIN protelomerase.

In one embodiment, the nucleic acid molecule as described herein comprises a nucleic acid construct as described herein, wherein the locations of: i) a nucleotide sequence encoding an adeno-associated virus (AAV) capsid variant wherein the nucleotide sequence is operably linked to a promoter for driving expression in an insect cell; ii) an enhancer element that is operably linked to the promoter, wherein the enhancer element is dependent on a transcriptional transregulator, and wherein introduction of the transcriptional transregulator into an insect cell comprising the nucleic acid construct, induces transcription from the promoter; iii) at least one AAV inverted terminal repeat (ITR) sequence; iv) optionally, a reporter gene operably linked to a promoter for driving expression in a mammalian cell; v) optionally, a barcode; and, the recognition sequence for the endonuclease in the construct are such that expression of AAV Rep proteins in an insect cell comprising the nucleic acid construct, effects replication and packaging into AAV capsids of a DNA fragment comprising elements i) to v).

In a third aspect, there is provided a library comprising a plurality of nucleic acid molecules comprising a nucleic acid construct as described herein. In one embodiment, the library comprises a plurality of nucleic acid molecules comprising a nucleic acid construct as described herein, wherein, in each member of the library, the nucleotide sequence encoding the AAV capsid variant differs by at least one nucleotide from nucleotide sequences encoding the AAV capsid variants in other members in the library. In one embodiment, the library comprises a plurality of nucleic acid molecules comprising a nucleic acid construct as described herein, wherein, in each member of the library, the nucleotide sequence encoding the AAV capsid variant encodes a capsid variant that differs by at least one amino acid from AAV capsid variants encoded by other members in the library.

In one embodiment, a nucleotide sequence encoding an AAV capsid variant in the library encodes an AAV capsid variant comprising a mutation (e.g., insertion, deletion and/or substitution) of one or more amino acids.

In one embodiment, a nucleotide sequence encoding an AAV capsid variant in the library encodes an AAV capsid variant comprising at least one amino acid difference (mutation) as compared to a wild type AAV capsid protein. A wild type AAV capsid protein is herein understood as an AAV capsid protein having the amino acid sequence of a naturally occurring AAV isolate, e.g., a clinical isolate.

In one embodiment, a nucleotide sequence encoding an AAV capsid variant in the library encodes an AAV capsid variant comprising at least one amino acid difference (mutation) as compared to a wild type AAV capsid protein, in a hypervariable and/or surface-exposed loop of the capsid protein variant.

In one embodiment, a nucleotide sequence encoding an AAV capsid variant in the library encodes an AAV capsid variant comprising at least one amino acid difference (mutation) as compared to a wild type AAV capsid protein, in a VR region of a surface loop of the capsid protein variant, including in at least one of VR-I, VR-II, VR-III, VR-IV, VR-V, VR-VI, VR-VII, VR-VIII and VR-IX, of which VR-IV and VR-VIII are preferred and VR-VIII is more preferred.

In one embodiment, the above described at least one amino acid difference (mutation) as compared to a wild type AAV capsid protein comprises an insertion of a peptide. The inserted peptide can comprise 2, 3, 4, 5, 6, 7, 8, 9, 10, 11, 12, 13, 14, 15 or more amino acids.

In one embodiment, the above described at least one amino acid difference (mutation) as compared to a wild type AAV capsid protein is introduced into the AAV capsid proteins VP1, VP2 or VP3, or in two of the capsid proteins in any combination, or in all three. In some embodiments, the mutations are introduced into VP1. In some embodiments, the mutations are introduced into VP2. In some embodiments, the mutations are introduced into VP3. In some embodiments, the mutations are introduced into VP1 and VP2. In some embodiments, the mutations are introduced into VP1 and VP3. In some embodiments, the mutations are introduced into VP2 and VP3. In some embodiments, the mutations are introduced into VP1, VP2, and VP3.

In one embodiment, the above described at least one amino acid difference (mutation) as compared to a wild type AAV capsid protein is a single mutation (e.g., an insertion, a deletion and/or a substitution of an amino acid) introduced at a single site in a capsid protein's amino acid sequence, while in other embodiments, more than 1, 2, 3, 4, 5, 6, 7, 8, 9 10, 15, 20, 25, 30, 40, 50, 100 or more (including any number between 1 and 100 or more) mutations (e.g., insertions, deletions and/or substitutions) are introduced in the amino acid sequence of a capsid protein.

In one embodiment, a nucleotide sequence encoding an AAV capsid variant in the library encodes an AAV capsid variant comprising at least one amino acid difference (mutation) as compared to a wild type AAV capsid protein, whereby wild type AAV capsid protein can be a capsid protein of a serotype selected from the group consisting of AAV1, AAV2, AAV3A, AAV3B, AAV4, AAV5, AAV6, AAV7, AAV8, AAV9, AAV10, AAV11, AAV12, AAV13 and AAVrh10. In a further embodiment, the wild type AAV capsid protein can be a capsid protein of a Clade A, Clade B, Clade C, Clade D, Clade E or Clade F AAV isolate as defined by Gao et al. (2004, J. Virol. 78:6381-6388), exemplary sequences of which are disclosed in WO2005/033321 and are available from GenBank® Database with Accession No.'s AY530553 to AY530629.

In a fourth aspect, there is provided an insect cell comprising a nucleic acid molecule comprising a nucleic acid construct as described herein or comprising a library comprising (plurality) of such nucleic acid molecules comprising a nucleic acid construct as described herein.

In one embodiment, the insect cell can be any cell that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including (r)AAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA). A preferred insect cell according to the invention is an insect cell for production of recombinant parvoviral vectors, more specifically recombinant AAV vectors.

Growing conditions for insect cells in culture, and production of heterologous products in insect cells in culture are well-known in the art and described e.g. in the above cited references on molecular engineering of insect cells (see also WO2007/046703).

In one embodiment, an insect cell, comprising a nucleic acid molecule as described herein or comprising a library as described herein, further comprising a nucleic acid construct comprising at least one expression cassette for expression of AAV Rep proteins in the insect cell and a nucleic acid construct comprising at least one expression cassette for expression of the transcriptional transregulator (e.g., a baculoviral immediate-early protein (IE1) or its splice variant (IE0)). In a preferred embodiment, a single nucleic acid construct comprises both the expression cassette for expression of AAV Rep proteins and the expression cassette for expression of the transcriptional transregulator. More preferably, the single nucleic acid construct is a baculoviral vector comprising at least one expression cassette for expression of AAV Rep proteins in the insect cell, as the baculoviral vector already comprises at least one expression cassette for expression of a baculoviral immediate-early protein (IE1) and/or its splice variant (IE0).

AAV replicases are non-structural proteins encoded by the rep gene. In wild type parvoviruses the rep gene produces two overlapping messenger ribonucleic acids (mRNA) with different length, due to an internal P19 promoter. Each of these mRNA can be spliced out or not to eventually generate four Rep proteins, Rep78, Rep68, Rep52 and Rep40. The Rep78/68 and Rep52/40 are important for the ITR-dependent AAV genome or transgene replication and viral particle assembly. Rep78/68 serve as a viral replication initiator proteins and act as replicase for the viral genome (Chejanovsky and Carter, J Virol., 1990, 64:1764-1770; Hong et al., Proc Natl Acad Sci USA, 1992, 89:4673-4677; Ni., et al., J Virol., 1994, 68:1128-1138). The Rep52/40 protein is DNA helicase with 3′ to 5′ polarity and plays a critical role during packaging of viral DNA into empty capsids, where they are thought to be part of the packaging motor complex (Smith and Kotin, J. Virol., 1998, 4874-4881; King, et al., EMBO J., 2001, 20:3282-3291). To produce AAV from the baculoviral vectors in an insect cell platform, the presence of both Rep68 and Rep40 is not prerequisite (Urabe, et al., 2002).

A nucleotide sequence encoding an AAV Rep protein or encoding AAV Rep proteins, is herein understood as a nucleotide sequence encoding at least one of the two non-structural Rep proteins, Rep 78 and Rep52, that together are required and sufficient for parvoviral vector production in insect cells. The AAV nucleotide sequence preferably is from a human or simian AAV and most preferably from an AAV which normally infects humans (e.g., serotypes 1, 2, 3A, 3B, 4, 5, 6, 8 and 9) or primates (e.g., serotypes 1 and 4). Examples of nucleotide sequences encoding parvoviral Rep proteins are given in SEQ ID NO's: 3-9.

It is understood that the exact molecular weights of the Rep78 and Rep52 proteins, as well as the exact positions of the translation initiation codons may differ between different parvoviruses. However, the skilled person will know how to identify the corresponding position in nucleotide sequence from other parvoviruses than AAV-2. Preferably, the nucleotide sequence encodes parvovirus Rep proteins that are functionally active in the sense that they have the required activities of viral replication initiator protein, replicase of the viral genome, DNA helicase and packaging of viral DNA into empty capsids as described above, sufficient for parvoviral vector production in insect cells. In one embodiment, possible false translation initiation sites in the Rep protein coding sequences, other than the Rep78 and Rep52 translation initiation sites are eliminated. In one embodiment, putative splice sites that may be recognised in insect cells are eliminated from the Rep protein coding sequences. Elimination of these sites will be well understood by an artisan of skill in the art.

In one embodiment, the nucleic acid construct for expression of the parvoviral Rep proteins comprises a single expression cassette for expression of at least both the parvoviral Rep78 and Rep52 proteins. In one embodiment, the single expression cassette for expression of at least both the parvoviral Rep78 and Rep52 proteins comprises a single open reading frame encoding at least both the parvoviral Rep78 and Rep52 proteins and having a suboptimal translation initiation codon for the Rep78 coding sequence, which suboptimal initiation codon effect partial exon skipping so that both at least both the parvoviral Rep78 and Rep52 proteins are translated in the insect cell, as e.g. described in U.S. Pat. No. 8,512,981, incorporated herein by reference. Suitable suboptimal translation initiation codons include e.g. ACG, CTG, TTG and GTG. In another embodiment, the single expression cassette for expression of at least both the parvoviral Rep78 and Rep52 proteins comprises in 5′ to 3′ order: (i) a first promoter linked operably to a 5′ portion of a first open reading frame of a parvovirus Rep78 protein, the first open reading frame comprising a translation initiation codon, (ii) an intron comprising a second insect cell promoter, the second promoter operably linked to a 5′ portion of an at least one additional open reading frame of a parvovirus Rep52 gene, wherein the at least one additional open reading frame comprises at least one additional translation initiation codon and overlaps with the 3′ portion of the first open reading frame, e.g. described in U.S. Pat. No. 8,945,918, incorporated herein by reference.

In another embodiment, the nucleic acid construct for expression of the parvoviral Rep proteins comprises at least two separate expression cassettes, one for expression of at least a parvoviral Rep78 protein and another for expression of at least a parvoviral Rep52 protein. Preferably, in this embodiment, the parvoviral Rep78 protein and the parvoviral Rep 52 protein comprise a common amino acid sequence comprising the amino acid sequence from the second amino acid to the most C-terminal amino acid of the parvoviral Rep 52 protein, wherein the common amino acid sequences of the parvoviral Rep78 protein and the parvoviral Rep52 protein are at least 90, 91, 92, 93, 94, 95, 96, 97, 98, 99 or 100% identical, and wherein the nucleotide sequence encoding the common amino acid sequence of the parvoviral Rep78 protein and the nucleotide sequence encoding the common amino acid sequences of the parvoviral Rep52 protein are less than 90, 89, 88, 87, 86, 85, 84, 83, 82, 81, 80, 79, 78, 77, 76, 75, 74, 73, 72, 71, 70, 69, 68, 67, 66, 60% identical, such as is described in U.S. Pat. No. 8,697,417, incorporated herein by reference. In a further embodiment, the nucleotide sequence encoding the common amino acid sequence of the parvoviral Rep78 protein has an improved codon usage bias for the cell as compared to the nucleotide sequence encoding the common amino acid sequences of the parvoviral Rep52 protein. Preferably, however, the nucleotide sequence encoding the common amino acid sequence of the parvoviral Rep52 protein has an improved codon usage bias for the cell as compared to the nucleotide sequence encoding the common amino acid sequences of the parvoviral Rep78 protein. Preferably, the difference in codon adaptation index (as defined hereinabove) between the nucleotide sequences coding for the common amino acid sequences in the parvoviral Rep78 protein and the parvoviral Rep52 protein is at least 0.1, 0.2, 0.3, 0.4, 0.5, 0.6, 0.7, 0.8, 0.9 or 1.0 whereby more preferably, the CAI of the nucleotide sequence coding for the common amino acid sequence in the parvoviral Rep52 protein is at least 0.5, 0.6, 0.7, 0.8, 0.9 or 1.0.

In one embodiment, the nucleotide sequences coding for the parvoviral Rep78 protein is SEQ ID NO: 9, coding for the wt AAV Rep78 protein and the nucleotide sequences coding for the parvoviral Rep52 selected from one of SEQ ID NO's: 4-7, each of which has been modified to have a different codon usage than the wild type Rep78 coding sequence of SEQ ID NO: 9. In a preferred embodiment, the nucleotide sequence coding for the parvoviral Rep78 protein is SEQ ID NO: 9, and is used in combination SEQ ID NO: 7 as nucleotide sequence coding for the parvoviral Rep52, the latter having been modified to differ as much as possible from SEQ ID NO: 9 in codon usage.

In one embodiment, the two separate expression cassettes for resp, the Rep78 and Rep52 proteins in the insect cell are optimised to obtain a desired molar ratio of the Rep78 to Rep52 proteins in the cell. Preferably, the combination of Rep78 and Rep52 expression cassettes in the cell produces a molar ratio of Rep78 to Rep52 in the range of 1:10 to 10:1, 1:5 to 5:1, or 1:3 to 3:1 in the (insect) cell. More preferably, the combination of Rep78 and Rep52 expression cassettes produces a molar ratio of Rep78 to Rep52 that is at least 1:2, 1:3, 1:5 or 1:10. The molar ratio of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50). A desired molar ratio of Rep78 to Rep52 can be obtained by the choice of the promoters in respectively the Rep78 and Rep52 expression cassettes as herein further described below. Alternatively or in combination, the desired molar ratio of Rep78 to Rep52 can be obtained by using means to reduce the steady state level of the at least one of parvoviral Rep 78 and 52 proteins. Thus, in one embodiment, the nucleotide sequence encoding the mRNA for the parvoviral Rep protein comprises a modification that affects a reduced steady state level of the parvoviral Rep protein. The reduced steady state condition can be achieved for example by truncating the regulation element or upstream promoter (Urabe et al., supra, Dong et al., supra), adding protein degradation signal peptide, such as the PEST or ubiquitination peptide sequence, substituting the start codon into a more suboptimal one, or by introduction of an artificial intron as described in WO 2008/024998. When using the two separate expression cassettes for resp, the Rep78 and Rep52 proteins in the insect cell the promoter in the Rep52 cassette is preferably stronger than the promoter in the Rep78 cassette. In one embodiment, the promoters in resp, the Rep78 and Rep52 cassettes are baculoviral promoters. In one embodiment, the promoters in resp, the Rep78 and Rep52 cassettes are distinct. In one embodiment, the Rep78 promoter is a delayed early baculoviral promoter, such as the 39k promoter. In one embodiment, the Rep52 promoter is a late or very late baculovirus promoter, such as the polH, p10, p6.9 and pSel120 promoters. In one embodiment, the late or very late baculovirus promoter that is used in the Rep52 cassette is a different promoter than the promoter used in the above-defined nucleic acid constructs comprising an expression cassette for expression of the capsid proteins.

In a preferred embodiment, the nucleotide sequence encoding at least one of parvoviral Rep protein comprises an open reading frame that starts with a suboptimal translation initiation codon. The suboptimal initiation codon preferably is an initiation codon that affects partial exon skipping. Partial exon skipping is herein understood to mean that at least part of the ribosomes do not initiate translation at the suboptimal initiation codon of the Rep78 protein but may initiate at an initiation codon further downstream, whereby preferably the (first) initiation codon further downstream is the initiation codon of the Rep52 protein. Alternatively, the nucleotide sequence encoding a parvoviral Rep protein comprises an open reading frame that starts with a suboptimal translation initiation codon and has no initiation codons further downstream. The suboptimal initiation codon preferably affects partial exon skipping upon expression of the nucleotide sequence in an insect cell. Preferably, the suboptimal initiation codon affects partial exon skipping in an insect cell so as to produce in the insect cell a molar ratio of Rep78 to Rep52 in the range of 1:10 to 10:1, 1:5 to 5:1, or 1:3 to 3:1. The molar ratio of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50).

The term “suboptimal initiation codon” herein not only refers to the tri-nucleotide initiation codon itself but also to its context. Thus, a suboptimal initiation codon may consist of an “optimal” ATG codon in a suboptimal context, e.g. a non-Kozak context. However, more preferred are suboptimal initiation codons wherein the tri-nucleotide initiation codon itself is suboptimal, i.e. is not ATG. Suboptimal is herein understood to mean that the codon is less efficient in the initiation of translation in an otherwise identical context as compared to the normal ATG codon. Preferably, the efficiency of suboptimal codon is less than 90, 80, 60, 40 or 20% of the efficiency of the normal ATG codon in an otherwise identical context. Methods for comparing the relative efficiency of initiation of translation are known per se to the skilled person. Preferred suboptimal initiation codons may be selected from ACG, TTG, CTG, and GTG. More preferred is ACG. A nucleotide sequence encoding parvovirus Rep proteins, is herein understood as a nucleotide sequence encoding the non-structural Rep proteins that are required and sufficient for parvoviral vector production in insect cells such the Rep78 and Rep52 proteins.

In a fifth aspect, there is provided a library comprising a plurality of AAV capsid variants, wherein members in the library comprise a nucleic acid that is replicated and packaged from a member in the library of nucleic acid molecules comprising a nucleic acid construct as described herein, e.g. as described in the third aspect. The library thus comprises or consists of a plurality of of AAV vector virions comprising the plurality of AAV capsid variants and the nucleic acid constructs. Each member of the library thus comprises an AAV capsid variant comprising a mutation (e.g., insertion, deletion and/or substitution) of one or more amino acids. Preferably, each member of the library comprises an AAV capsid variant comprising a mutation as compared to a wild type AAV capsid protein. The mutations in the capsid variants as defined herein above. In one embodiment, the library comprises at least 10⁴, 10⁵, 10⁶, 10⁷, 10⁸ or more different AAV capsid variants genes.

In a sixth aspect, there is provided a method for preparing a library of AAV capsid variants. In one embodiment, the method for preparing a library of AAV capsid variants comprises the steps of: a) providing a library comprising a plurality of nucleic acid construct as defined herein above, wherein the amino acid sequence of an AAV capsid variant of each member in the library as encoded in the construct of that member, differs by at least one amino acid from amino acid sequences of AAV capsid variants encoded in constructs of other members in the library; b) amplifying the library of the nucleic acid constructs obtained in a), preferably by rolling circle amplification; c) digestion of the amplified library of the nucleic acid constructs obtained in b) by a site-specific endonuclease that cuts at a recognition sequence in the nucleic acid construct to produce a library comprising linearised amplified nucleic acid constructs of unit length; d) transfection of the library comprising linearised amplified nucleic acid constructs obtained in c) into insect cells; e) infection of the insect cells with a baculoviral vector comprising at least one expression cassette for expression of AAV Rep proteins in the insect cells and culturing of the cells; and, f) recovery of a library of AAV capsid variants produced by the insect cells in e).

In one embodiment, the method for preparing a library of AAV capsid variants comprises providing a library comprising a plurality of nucleic acid construct as described herein, wherein, in each member of the library, the nucleotide sequence encoding the AAV capsid variant encodes a capsid variant that differs by at least one amino acid from AAV capsid variants encoded by other members in the library, whereby the AAV capsid variants and their differences can be as herein described above.

In one embodiment of the method for preparing a library of AAV capsid variants, the library in step a) is provided by: i) providing a nucleic acid construct as defined herein above (as template or original); and, ii) replacing a fragment comprising at least a part of the nucleotide sequence encoding the AAV capsid variant in the construct, by a library of fragments, wherein the amino acid sequence of an AAV capsid variant of each member in the library as encoded in the fragment of that member, differs by at least one amino acid from amino acid sequences of AAV capsid variants encoded in constructs of other members in the library. In one embodiment of the method, the replacement of the fragment comprising at least a part of the nucleotide sequence encoding the AAV capsid variant in the construct in step ii) is performed by Gibson assembly (Gibson et al., 2009, Nat Methods. 6(5):343-5). It is preferred to use in vitro recombinant DNA cloning methods such as Gibson assembly, so as to avoid a bias in the cloning of some library members over others, as may occur in biological host systems in vivo.

In one embodiment, the method for preparing a library of AAV capsid variants comprises amplifying the library of the nucleic acid constructs. “Amplification” used in reference to a nucleic acid or nucleic acid reactions, is herein understood to refer to in vitro methods of making copies of a particular nucleic acid or of a collection of nucleic acids, such as the nucleic acid construct members in the library. Numerous methods of amplifying nucleic acids are known in the art, and amplification reactions include polymerase chain reactions, ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278) and isothermal amplification reactions. In the method, the nucleic acid construct members in the library are amplified so as to obtain a sufficient amount of the nucleic acid construct members for transfection of the insect cells. It is preferred to use an in vitro amplification methods so as to avoid a bias in the amplification of some library members over others, as may occur in biological host systems in vivo. In one embodiment, the library is amplified by rolling circle amplification using a @29 DNA polymerase. The phi29 DNA polymerase is the replicative polymerase from the Bacillus subtilis phage phi29 (φ29), which has exceptional strand displacement and processive synthesis properties, and which has an inherent 3′→5′ proofreading exonuclease activity. In one embodiment, random primers, e.g. random hexamer primers, are used from priming the DNA polymerase, e.g. the φ29 DNA polymerase.

In one embodiment, the method for preparing a library of AAV capsid variants comprises digestion of the amplified library of the nucleic acid constructs obtained in b) by a site-specific endonuclease that cuts at a recognition sequence in the nucleic acid construct to produce a library linearised amplified nucleic acid constructs of unit length. The site-specific endonuclease cuts at recognition sequence for the endonuclease in the nucleic acid constructs, which recognition sequence preferably is unique within the nucleic acid constructs. The site-specific endonuclease, its recognition sequence and its location within the construct can be as described herein above.

In one embodiment, the method for preparing a library of AAV capsid variants is a method wherein in step c) the ends of the linearised amplified library of the nucleic acid constructs of unit length are provided with protection against exonuclease degradation. Protection against exonuclease degradation can be provided using methods and means as herein described above. In one embodiment, the site-specific endonuclease is a protelomerase, providing covalently closed ends to the linearised amplified library of the nucleic acid constructs of unit length. The protelomerase preferably is a phage N15 TeIN protelomerase. In this embodiment, the nucleic acid constructs will thus comprise recognition sequence for the protelomerase in question and at a location in the construct as herein described above.

In a seventh aspect, there is provided a method for identifying an AAV capsid variant with a desired characteristic. In one embodiment, the method comprises: a) providing a library of AAV capsid variants as defined herein above, or as obtained in a method for preparing a library of AAV capsid variants as defined herein above; b) contacting the library with a culture of cells, an organoid or a tissue, or administering the library to a non-human animal; c) allowing AAV capsid variants in the library to transduce the cells, the organoid, the tissue or cells in the animal; and, d) identifying at least one AAV capsid variant that has transduced at least one desired cell-type in the cells, the organoid, the tissue or in the animal in b), as an AAV capsid variant with the desired characteristic, and optionally recovery of the AAV capsid variant with the desired characteristic from the desired cell.

The method for identifying an AAV capsid variant with a desired characteristic as described herein, further comprises a step (b), wherein the library is contacted with a culture of cells, an organoid or a tissue, or wherein the library is administered to a non-human animal. In one embodiment, the culture of cells, an organoid or a tissue, or the non-human animal comprise at least one desired cell-type, e.g. cell-type for which an AAV capsid variant with improved tropism is to be identified. In one embodiment, the culture of cells, an organoid or a tissue, or the non-human animal can further comprise at least one non-desired cell-type, e.g. cell-type for which the AAV capsid variant to be identified has reduced transduction efficiency. In one embodiment, the library is systemically administered to a non-human animal, e.g. using an intravenous route of administration.

In a next step (c) of the method for identifying an AAV capsid variant with a desired characteristic as described herein, the AAV capsid variants in the library are allowed to transduce the cells, the organoid, the tissue or cells in the animal. Thus, in one embodiment of step (c), sufficient time is allowed to elapse for the AAV capsid variants in the library are allowed to transduce the (desired) cells, the organoid, the tissue or cells in the animal and to express the reporter gene. In a next step (d) of the method for identifying an AAV capsid variant with a desired characteristic as described herein, at least one AAV capsid variant that has transduced at least one cell of the desired cell-type in the cells, the organoid, the tissue or in the animal is identified. In one embodiment, the at least one AAV capsid variant is identified by sequencing its UMI. In one embodiment, the at least one AAV capsid variant is identified as an AAV capsid variant with the desired characteristic. In one embodiment, step (d) further comprises recovery of the AAV capsid variant with the desired characteristic from a cell of the desired cell-type.

In one embodiment, step (d) comprises detection of transduction of a cell of the desired cell-type by detecting expression of the reporter gene in at least one cell of the desired cell-type.

In one embodiment of the method for identifying an AAV capsid variant with a desired characteristic as described herein, the desired cell-type, e.g. cell-type for which an AAV capsid variant with improved tropism is to be identified, is a cell of an organ or tissue selected from liver, skeletal muscle, heart, diaphragm muscle, kidney, brain, stomach, intestines, skin, endothelial cells and lungs. In one embodiment of the method for identifying an AAV capsid variant with a desired characteristic as described herein, an AAV capsid variant with a desired characteristic to be identified has a reduced transduction efficiency of a non-desired cell-type, which non-desired cell-type can be a cell of an organ or tissue selected from liver, skeletal muscle, heart, diaphragm muscle, kidney, brain, stomach, intestines, skin, endothelial cells and lungs.

In one embodiment, the method for identifying an AAV capsid variant with a desired characteristic as described herein, is a method wherein in step d) in at least one cell of the desired cell-type, for at least two members in the library, at least one of the mRNA expressed from and the genome copy-number of the least two members is quantified and identified by sequencing its barcode/UMI, and wherein the member with at least one of the highest mRNA expression level and genome copy-number is identified as the AAV capsid variant with the desired characteristic. In one embodiment of the method, in step d), in cells of more than one cell-types including the at least desired cell-type, for at least two members in the library, at least one of the mRNA expressed from and the genome copy-number of the least two members is quantified and identified by sequencing its barcode/UMI, and wherein the member with the most desired distribution over the more than one cell-types is identified as the AAV capsid variant with the desired characteristic. In one embodiment, the cells of more than one cell-types also include at least a non-desired cell-type.

In one embodiment, the method for identifying an AAV capsid variant with a desired characteristic as described herein, is a method, wherein the variant with the desired characteristic is identified by sequencing at least one of i) the barcode/UMI and ii) a region of the DNA construct encoding the at least one amino acid difference.

The present invention has been described above with reference to a number of exemplary embodiments as shown in the drawings. Modifications and alternative implementations of some parts or elements are possible, and are included in the scope of protection as defined in the appended claims.

### DESCRIPTION OF THE FIGURES

FIG. 1. DNA scaffold and AAV production method by transfection/baculovirus infection. Replication of the transfected DNA scaffold is facilitated by the infection of a Rep Baculovirus through the AAV2 ITRs and hr4b enhancer. Furthermore the baculovirus co-infection transactivates the P10 promoter which drives expression of the capsid VP proteins. The capsids are then assembled in the cell, after which the replicated transgene get packaged into the capsids by the Rep protein, resulting in an AAV library.

FIG. 2. The transfection/baculovirus infection method results in low Total/full ratio AAV batches because only the cells that receive both components can produce AAV capsids and package transgenes in them.

FIG. 3. Grit process (Gibson assembly—Rolling Circle amplification—TelN digestion) of introducing variation, amplifying the library and generating suitable template for AAV library production with the transfection/baculovirus infection method.

FIG. 4.
A) AAV2/5 production with rolling circle/TelN digestion of an AAV2/5 scaffold and the transfection/infection methods. AAV2/5 scaffold gets amplified from plasmid pIMRAku04 by rolling circle amplification. Next linear, covalently closed scaffolds are created by TelN protelomerase digestion. Following transfection and baculovirus infection, AAV is harvested and analyzed by Q-PCR and SDS-Page.
B) AAV2/5 scaffold pIMRaku04. Elements present on the scaffold are; 1. AAV2 ITRs, 2. hr4b enhancer, 3. Nanoluc reporter cassette, 4. AAV2/5 capsid expression cassette under control of a P10 promoter and 5. TelN protelomerase digestion site.

FIG. 5. SDS Page gel run with purified AAV batches produced with either the pIMRaku-04 scaffold or pIMRaku-04 plasmid. The VP123 ratio of the AAV2/5 material produced with the pIMRaku-04 scaffold displays the natural stoichiometry of ~1:1:10 VP123 for AAV.

FIG. 6. Library DNA scaffolds pIMRAku-04 AAV2/5 and pIMRAku-011 with random 21 bp library inserted at VR-VIII. The insertion should result in the production of an AAV2/5 or AAV9 capsid library with an 7mer peptide insertion at the VR-VIII surface exposed loop. Elements present on the scaffold are as described previously in FIG. 4b.

FIG. 7. Viral titers in gc/ml of AAV2/5 (CLB=crude lysate)) and AAV9 (purified) libraries as measured with a QPCR specific for the hr4b enhancer element.

FIG. 8. SDS Page gel run with purified AAV2/5 and AAV9 libraries. The capsid stochiometry of both libraries displays the natural VP123 ratio of 1:1:10 for AAV.

FIG. 9. Sanger sequencing at VR-VIII library insertion site of AAV2/5 (A) and AAV9 (B). Sequencing intermediate samples taken while producing the AAV libraries reveals mixed read at the insertion sites throughout the production process.

FIG. 10. Maintenance of complexity during AAV2/5 library production process using NGS. Of the initial 1.22×10⁷ variants detected in the starter G-Block, 8.1×10⁶ variants get amplified by the GRIT process. 3.2×10⁶ variants are detected in the purified AAV2/5 library prep. Overall approximately 25% of the original complexity in the G-Block is maintained in the purified AAV2/5 library.

FIG. 11. AAV cross-packaging occurs when the genome and capsid are a mismatched during production. For example AAV5 capsid with AAV9 genome or AAV9 capsid with AAV5 genome.

FIG. 12. A: Simplified method of determining the cross-packaging rate in two capsid libraries produced in expresSF+ or HEK293t cells. AAV2/5 and AAV9f are co-produced in SF+ and Hek293t cells. Following sequential purification with serotype specific affinity resins, genome identity is established with a serotype specific QPCR. % cross-packaging is calculated by determining the fraction of mismatched genomes in the obtained pools. B: Percentage crosspackaging following purification with serotype specific resins (qPCR).

### EXAMPLES

### Introduction

AAV capsid libraries have been widely and successfully applied to select capsids with improved traits in directed evolutionary studies. Production of these AAV libraries leverages the mammalian production platform in Hek293 cells. Libraries produced on this platform have sufficient depth and titer to perform these type of studies. However, their production requires the transfection of high quantities of plasmid DNA, making them prone to transgene cross packaging. Cross packaging occurs when an AAV genome is packaged into a mismatched capsid. AAV libraries with a high degree of cross packaging will generate a significant amount of noise in the downstream analysis of directed evolutionary studies. This is because it cannot be guaranteed that the detected AAV genome is originating from a cross packaged or correctly packaged AAV genome. Therefore, multiple cycles through an in vitro or in vivo model will need to be performed to increase confidence that a detected genome (with potentially beneficial capsid properties) is originating from a correctly packaged AAV and not from a cross packaged AAV. These cycles can significantly add to the length of such studies increasing their costs and decreasing the feasibility of the directed evolutionary approach to be selected for new capsid development. Minimizing the degree of cross packaging of AAV libraries is therefore imperative.

The method we developed in this invention relates to the generation of AAV capsid libraries in insect cells. In house experiments showed that insect cells are highly sensitive to transfection with a high levels of exogenous DNA resulting in a self-limiting mechanism when delivering high quantities of DNA to the cell. The maximum amount of DNA which can be successfully delivered to an insect cells falls in the 1-2 μg DNA/cell range, well below the lower limit of DNA that is normally transfected to make AAV libraries in Hek293 (>5 μg DNA/cell). The AAV capsid library production method that we developed leverages this observation and combines it with the transactivation and transgene replication capabilities that are brought to the insect cell by a co-infection with a Replicase (Rep) baculovirus.

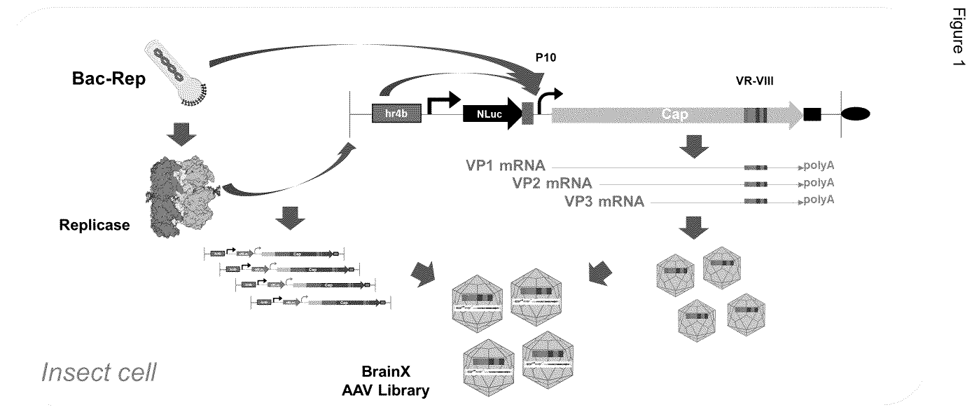

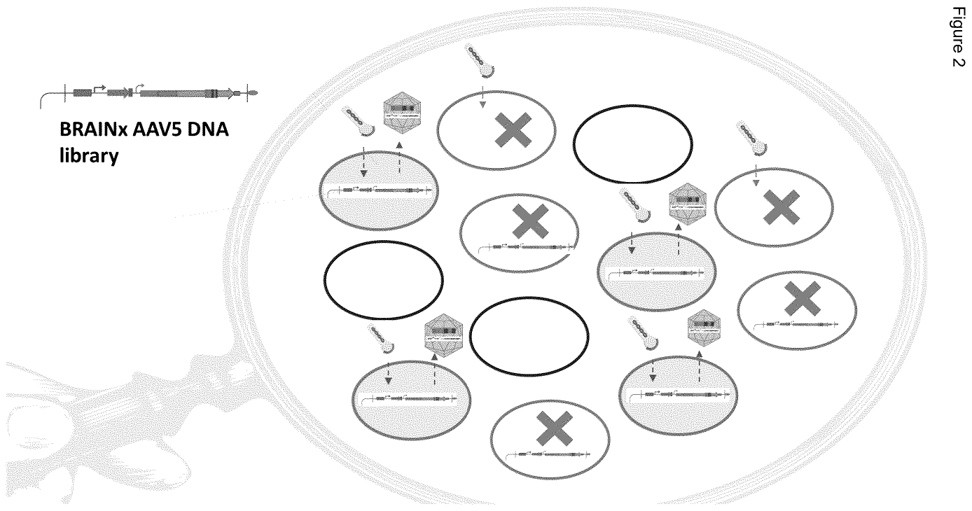

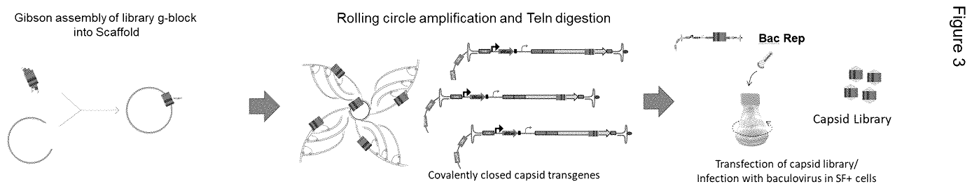

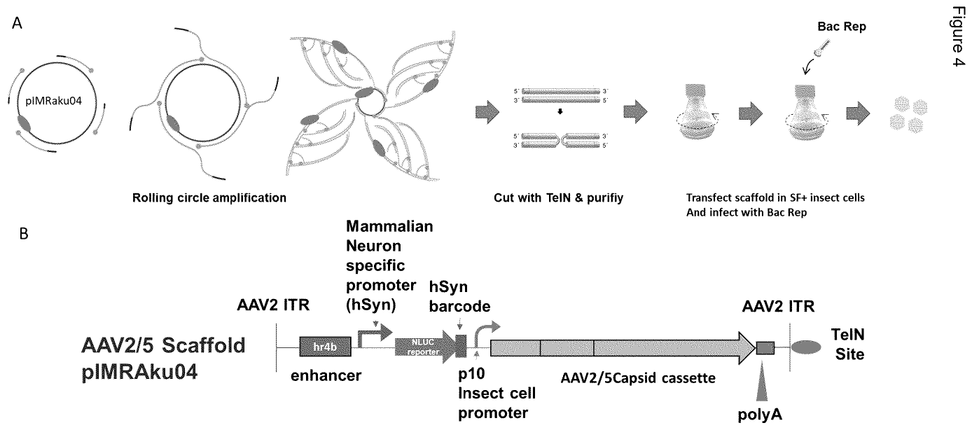

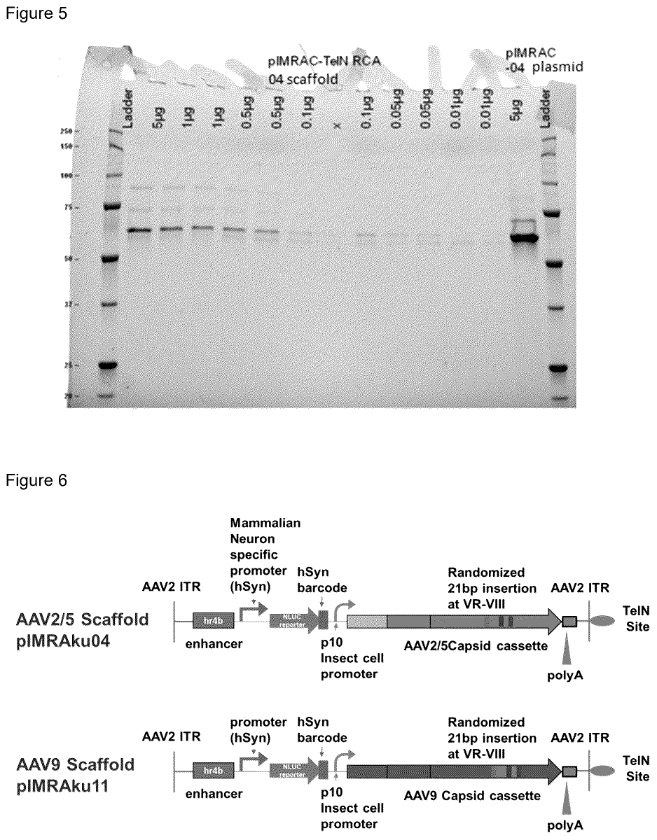

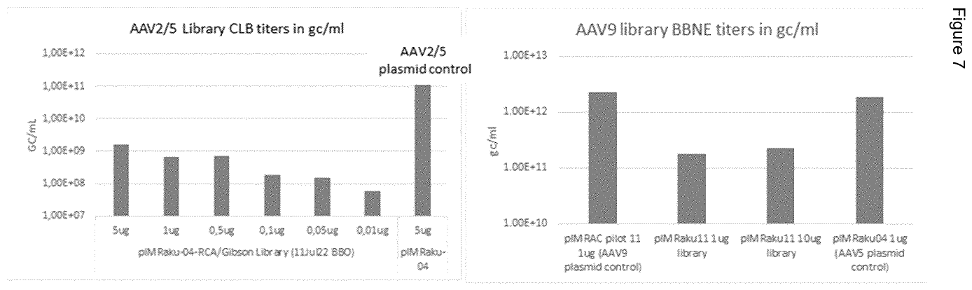

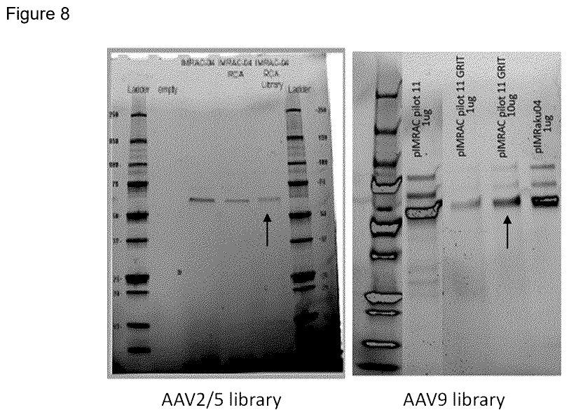

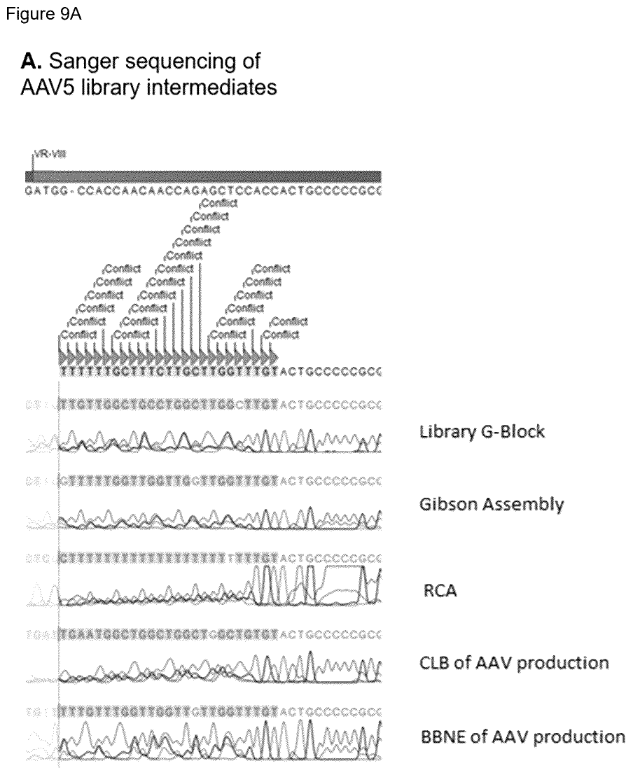

We designed a DNA scaffold that facilitates the production of high titer AAV in insect cells with a low amount of DNA delivered to the cell by transfection (<1 μg/cell). Following transfection of the DNA scaffold, the cell is co-infected with a Rep containing baculovirus. In the cell, the Rep baculovirus facilitates the replication of the DNA scaffold (via the AAV2 ITRs and hr4b enhancer) and transactivation of the promoter (P10) that drives expression of the AAV capsid proteins (FIG. 1). An additional advantage of this production method is that the produced AAV has a low degree of empty capsids. This is because only cells that are both transfected with the DNA scaffold and infected by the Rep baculovirus will produce AAV (FIG. 2).

Regardless of the production platform (mammalian or insect cells), AAV libraries need a method of introducing variation into the AAV capsid gene. In order to develop deep and complex libraries this introduced variation will need to be maintained throughout the different AAV production process steps (eg. variation introduction into the scaffold, Library DNA scaffold amplification, AAV production and purification). The libraries produced in these Examples introduce complexity to the capsid genes by inserting randomized 21 bp DNA sequences into surface exposed loop VR-VIII of the capsid gene. We developed a three step process called GRIT (Gibson assembly—Rolling circle amplification—TelN proteolomerase digestion) that can introduce variation as well as amplify the DNA library scaffold. The GRIT process is capable of maintaining complexity of the introduced insertions from insertion through to amplification of the DNA library scaffold.

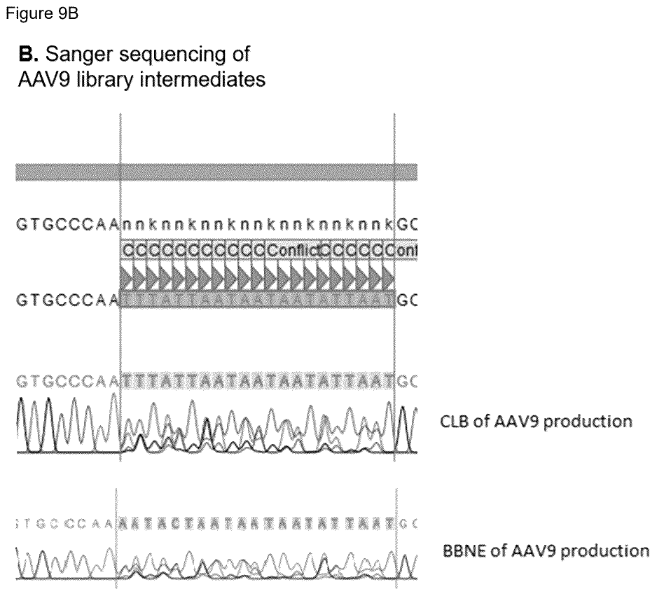

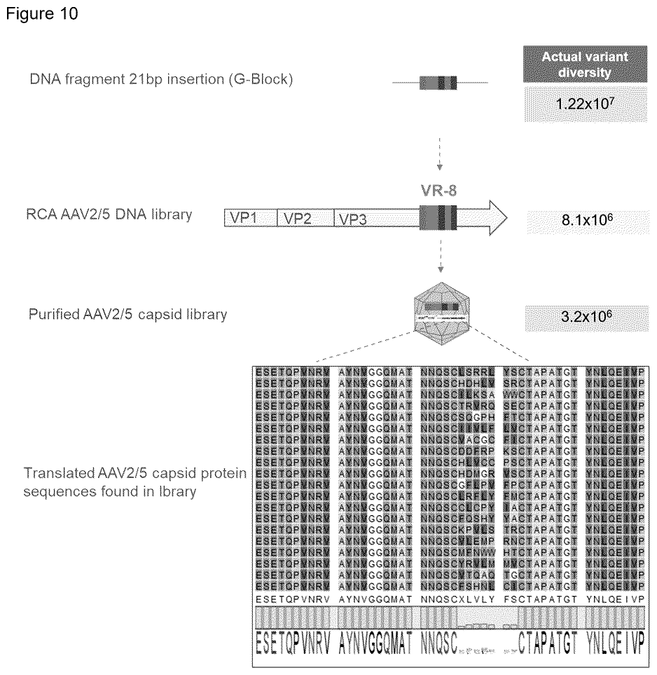

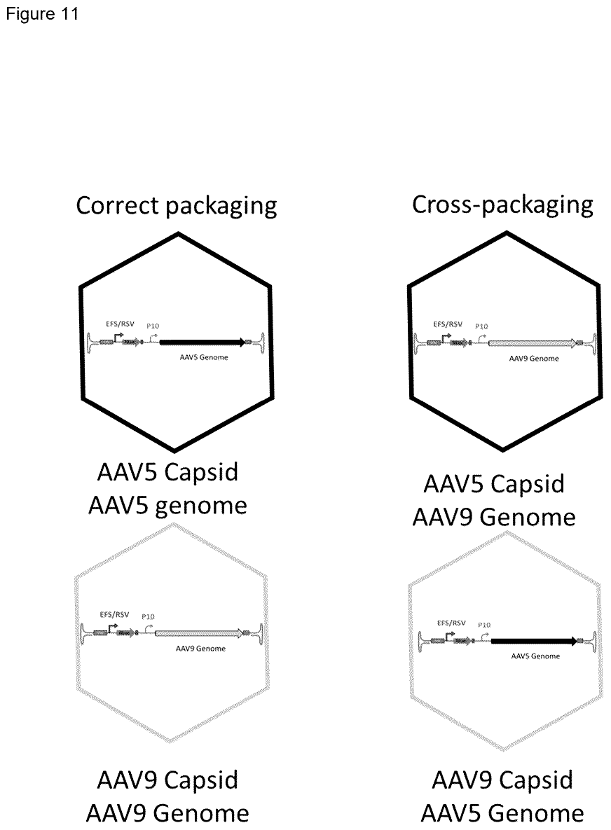

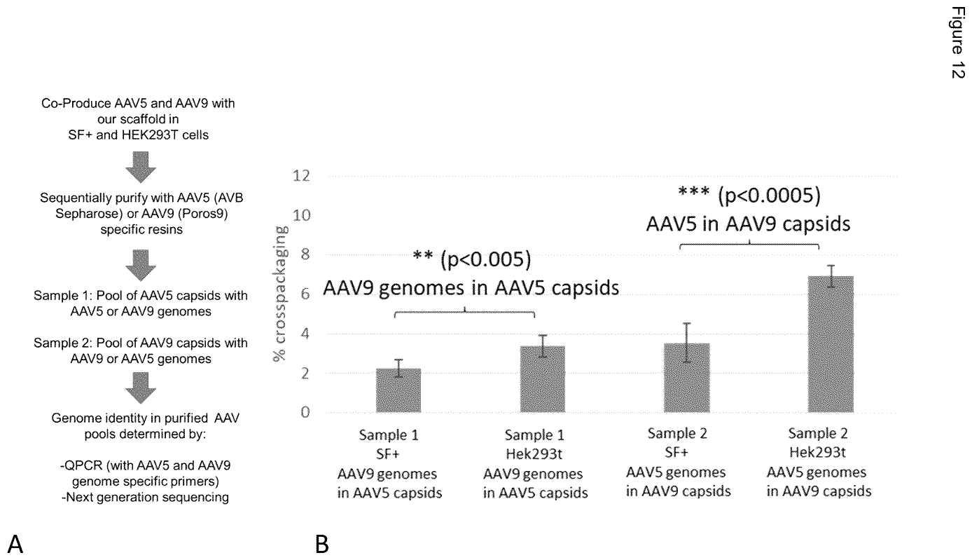

In brief, a library of 21 bp long randomized DNA sequences (encoding for 7-mer peptides) is inserted into the linearized DNA scaffold by Gibson assembly. The Gibson assembly recirculates the DNA library scaffold allowing it to the amplified by rolling circle amplification with the phi29 enzyme. Covalently closed singular library DNA scaffolds are generated from the amplified template by TelN protelomerase digestion, generating linear cassettes. The main advantage of a covalently closed scaffold is that it enhances the transfection efficiency of the scaffold versus covalently open linear DNA. Ultimately, library DNA scaffolds can be transfected into insect cells to produce the AAV capsid library (FIG. 3).

In the first Example we investigate the feasibility of using a rolling circle amplified/Teln digested AAV2/5 DNA scaffold without a library for the production of AAV2/5 batches in insect cells. In the second Example we evaluate the GRIT process for the production of an AAV2/5 library with a randomized 7-mer peptide insertions at VR-VIII of AAV2/5 in insect cells. AAV2/5 is an AAV5 chimera in which the VP1 originates from AAV2 and VP2/VP3 originates from AAV5. In addition we evaluate the applicability of the GRIT process/transfection infection method for the AAV9 serotype were we also introduce randomized 21 bp DNA sequence insertions at VR-VIII of the AAV9 gene and produce a library in insect cells.

### 1. Example 1: AAV Production with an AAV2/5 DNA Scaffold Amplified by Rolling Circle Amplification and Digested with TeIN Protelomerase

### 1.1 Methods

### 1.1.1 AAV5 DNA Scaffold Generation by Rolling Circle Amplification and TeIN Digestion

Plasmid pIMRaku-04 (AAV2/5, without a library; SEQ ID NO. 10) was amplified by rolling circle amplification (RCA) using EquiPhi29 enzyme (ThermoFisher). 10 ng of plasmid was amplified for 3 hours at 43° C. in the presence of 100 μM exoresistant random Hexamers (ThermoFisher) and 1 mM dNTP (NEB). After amplification the DNA was precipitated for 15 minutes at 21000×g in the presence of 0.1× final volume 3 M Potassium Acetate pH=5.2 and 0.7× final volume 100% isopropanol, followed by a wash for 5 minutes at 21000×g with 70% Ethanol. Ultimately, the DNA pellet was dissolved in 5 mM Tris HCl pH=8.5. Next, the long linear strands of RCA amplified DNA were digested into covalently closed DNA scaffolds flanked by ITRs using TeIN Protelomerase (NEB). Digestions were performed at 30° C. using 1 μg amplified DNA per μl of enzyme. Following digestion, the short linear DNA was column purified with a PCR purification kit (Machery-Nagel). DNA concentrations of the purified DNA scaffolds were determined on a Nanodrop 100 system (Thermofisher).

1.1.2 Production of AAV2/5 (plMRaku-04) Using Transfection/Baculovirus Infection Method in expresSF+ Insect Cells

Multiple concentrations of RCA amplified/TelN digested pIMRaku-04 scaffold ranging from 0.01 μg to 5 μg were transfected into 1.5×10⁷ expresSF+ cells using cellfectin II transfection reagent (ThermoFisher). A circular plasmid control was taken along in the experiment as well. To transfect cells with equal amounts of DNA, every condition was normalized to 15 μg of DNA with UltraPure Herring Sperm carrier DNA (ThermoFisher). 72 hours after the transfection, fresh baculovirus expressing AAV2 Replicase (SEQ ID NO. 14) was inoculated into the cells at 1% of the final volume (40 ml). 72 hours after the baculovirus infection, the AAV batches were harvested by adding 10× lysis buffer (1.5 M NaCl, 0.5 M Tris-HCl, 1 mM MgCl₂, 10% Triton X-100, pH=8.5) and lysing for 1 hour at 28° C. Genomic DNA was digested by Benzonase treatment for 1 hour at 37° C. Cell debris was removed by centrifugation for 15 minutes at 1900×g after which the supernatant containing the AAV2/5 was stored at 4° C. AAV2/5 particles were purified from crude lysates with a batch binding protocol using AVB sepharose affinity resin (GE Healthcare). AAV crude cell lysates were added to washed (with 0.2 M HPO₄ pH=7.5 buffer) resin. Subsequently, samples were incubated for 2 hours at room temperature under gentle mixing. Following the binding the resin was washed in 0.2 M HPO₄ PH=7.5 buffer and bound vectors were eluted by the addition of 0.2 M Glycine pH=2.5. The pH of the eluted vectors was immediately neutralized by the addition of 0.5 M Tris-HCl PH=8.5. Purified AAV batches were stored at −20° C.

### 1.1.3 Analysis of AAV Batches by QPCR and SDS PAGe Gel Electrophoresis

Titers of lysed AAV2/5 batches were determined with a Q-PCR specific for the hr4b enhancer region on the scaffold. AAVs were treated with DNAse at 37° C. to degrade exogenous DNA. AAV DNA was then released from the particles by a short (30 min) heat treatment (37° C.) in the presence of 1 M NaOH. Next the alkaline environment was neutralized by the addition of an equal volume of 1 M HCl. Neutralized DNA was diluted 10× in WFI 16 ng/μl PolyA, after which the samples were used in a QPCR with primers specific for the hr4b element on the transgene; forward CGAGGGATGATGTCATTTGTAGA (SEQ ID NO. 17), reverse ATCCACCGATCTTGCGTTAC (SEQ ID NO. 18) and probe Fam-ACCGAACTCGCTTTACGAGTAGAATTCT-mgb (SEQ ID NO. 19). VP protein composition of purified AAV2/5 vectors were determined by SDS page gel electrophoresis. Briefly, 15 μl of purified AAV2/5 was mixed with 5 μl 4× Leammli loading buffer (Biorad) supplemented with B-mercaptoethanol (Biorad). Following a short heat treatment to denature the proteins (5 min at 95° C.) material was loaded on a Stain free polyacrylamide gel (Biorad). Next, the samples were electrophoretically separated for 35 minutes at 200 Volts. Following electrophoresis stain (tryptophan based) was developed under UV-light for 5 minutes after which VP proteins were then visualized under UV light on a Chemidoc imaging system (Biorad).

### 1.2 Results

In this Example we investigate if the DNA scaffold we designed for producing AAV libraries in insect cell is capable of producing high titer AAV with the correct capsid viral protein stoichiometry of 1:1:10. A VP123 stoichiometry of approximately 1:1:10 reflects an infective capsid. To produce AAV, construct plMRAku04 (AAV2/5; SEQ ID NO. 10) was amplified by rolling circle amplification using EquiPhi29 and linearized with TelN protelomerase. The linearized pIMRaku04 scaffold was transfected into expresSF+ insect cells at concentrations ranging from 0.01 μg to 5 μg/reaction. Three days after transfection the cells were infected with 1% v/v AAV2 Rep expressing baculovirus (SEQ ID NO. 14). Following harvest, the AAV2/5 particles were purified with a batch binding protocol using AVB Sepharose (FIG. 4a).

For the experiment we used DNA scaffold pIMRAku04, which contains elements needed for the production of AAV2/5 capsids. These elements are: 1. A “to be packaged” AAV transgene cassette flanked by two AAV2 ITRs. The ITRs facilitate replication of the scaffold in the insect cell by Rep as well as its packaging into the AAV2/5 particle. 2. A Nanoluc reporter gene under control of a CNS specific promoter and flanked by a barcode. The promoter driving expression of the reporter can be swapped out depending on the tissue type for which a library is to be developed (such as Liver, CNS or heart specific). 3. AAV2/5 capsid expression cassette under control of the P10 promoter (SEQ ID NO. 11). Baculovirus infection enhances the activity of the P10 promoter. 4. Hr4b enhancer element, which is involved in enhancing the replication of the scaffold once in the cell as well in transactivating the P10 promoter driving capsid protein expression. 5. TeIN protelomerase cut site used for linearizing and covalently closing the DNA scaffold outside of the cassette flanked by the ITRs (FIG. 4b).

After production, AAV titers were determined in the crude lysed bulk (CLB) with a Q-PCR specific for the hr4b element (Table 1). We found a measurable AAV titer with all conditions using our pIMRAku04 scaffold. Furthermore we saw an increase in AAV titers depending on the amount of DNA scaffold transfected. Compared to a plasmid transfection the linearized/covalently closed pIMRAku04 scaffold produces approximately 1-log less efficient (3.29e+11 for 5 μg plasmid vs 3.70e+10 for 5 μg linearized scaffold). To investigate if the AAV2/5 capsids produced with the pIMRAku04 scaffold contained the correct VP stoichiometry we performed SDS-PAGE gel electrophoresis on batch binding purified material (FIG. 6). VP1/2/3 ratio of the AAV2/5 material produced with the method has a natural ratio of approximately 1:1:10, reflective of an infective AAV capsid.

Taken together the DNA scaffold that we designed is capable of producing relatively high titer AAV batches with the natural VP123 protein ratio ~1:1:10 reflective of an infective capsid. While AAV titers produced with the pIMRaku04 scaffold are approximately 1 log lower versus the plMRaku04 plasmid, the amount of virus produced per ml of CLB is sufficient for further library development with this scaffold design. This is because AAV libraries used for in vivo directed evolution studies are usually injected at relatively low concentrations of <5×10¹² gc/kg. These level that are within reach with the productivity of this AAV production method (when transfecting in the 0.5-2 μg scaffold DNA range).

TABLE 1
Transfection conditions of AAV2/5 scaffold pIMRaku-04 in insect
cells as well as the resulting AAV titers in crude lysates,
as measured by Q-PCR specific for the hr4b promoter.

| | (μg) used for | | Titer in CLB | CLB |
|---|---|---|---|---|
| Transfected | transfection | | (GC/mL) | (total GC) |
| pIMRaku-04-RCA/Teln | 5 | μg | 3.70e+10 | 9.25e+11 |
| (Scaffold) | 1 | μg | 1.86e+10 | 4.65e+11 |
| | 1 | μg | 1.58e+10 | 3.95e+11 |
| | 0.5 | μg | 1.25e+10 | 3.13e+11 |
| | 0.5 | μg | 8.16e+9 | 2.04e+11 |
| | 0.1 | μg | 3.31e+9 | 8.28e+10 |
| | 0.1 | μg | 3.53e+9 | 8.83e+10 |
| | 0.05 | μg | 1.61e+9 | 4.03e+10 |
| | 0.05 | μg | 1.52e+9 | 3.80e+10 |
| | 0.01 | μg | 3.93e+8 | 9.83e+9 |
| | 0.01 | μg | 4.09e+8 | 1.02e+10 |
| pIMRaku-04 | 5 | μg | 3.33e+11 | 8.33e+12 |
| (plasmid) | 5 | μg | 3.29e+11 | 8.23e+12 |

### 2. Example 2: Production of AAV2/5 and AAV9 Libraries with Randomized 7Mer Peptide Insertions at Variable Loop VR-VIII

### 2.1 Methods

### 2.1.1 AAV2/5 and AAV9 VR-VIII Random 21 bp Library Scaffold Generation by Gibson Assembly, Rolling Circle Amplification and TeIN Digestion (GRIT)

Plasmid pIMRAku-11 (AAV9; SEQ ID NO. 12, comprising the AAV9 expression cassette SEQ ID NO. 13) was digested with Xagl (NEB) and BamHI (NEB) restriction enzymes. 50 ng of linearized plasmid was combined with 6 ng of G-Block fragment EcoNI-Cap9-VR8-7mer-BamHI (IDT) in a scaffold to G-block ratio of 1:2. The amount of DNA used based in the reaction was on the size of the fragment. Next, a Gibson assembly was performed for 2 hours at 50° C. using the NEBuilder HIFI DNA assembly kit (NEB). Plasmid pIMRAku-04 (AAV2/5) was digested with the Ncol restriction enzyme (NEB). 50 ng of the linearized pIMRAku-04 was combined with 6 ng Gblock-7mer AAV5 VIII (IDT) in a scaffold to G-block ratio of 1:2. Gibson assembly was performed for 2 hours at 50° C. using the NEBuilder HIFI DNA assembly kit (NEB). The resulting circularized plasmids containing either the AAV9 or AAV2/5 capsid libraries with randomized 21 bp insertions at variable loop VR-VIII of the capsid gene were used as template for a rolling circle amplification reaction.

10 ng of recircularized library plasmids for AAV2/5 or AAV9 were amplified for 3 hours at 43c in the presence of 100 M exoresistant random Hexamers (ThermoFisher) and 1 mM dNTP (NEB). After amplification the DNA was precipitated for 15 minutes at 21000×g in the presence of 0.1× final volume 3 M Potassium Acetate pH=5.2 and 0.7× final volume 100% isopropanol followed by a wash for 5 minutes at 21000 g with 70% Ethanol. Ultimately, the DNA pellet was dissolved in 5 mM Tris HCl pH=8.5. Next, the long linear strands of RCA amplified DNA were digested into covalently closed DNA scaffolds flanked by ITRs using TeIN Protelomerase (NEB). Digestions were performed at 30° C. using 1 μg amplified DNA per μl of enzyme. Following digestion, the short linear DNA was column purified with a PCR purification kit (Machery-Nagel). DNA concentrations of the purified DNA scaffolds were determined on a nanodrop 100 system (Thermofisher).

2.1.2 Production of AAV2/5 and AAV9 Randomized 7Mer Peptide Libraries Using Transfection/Baculovirus Infection Method in expresSF+ Insect Cells 1 μg of AAV2/5 (pIMRAku-04) or AAV9 (pIMRAku-11) DNA scaffold libraries was transfected into 1.5e7 expresSF+ cells using cellfectin II transfection reagent (ThermoFisher). 15 μg of total DNA was transfected into the cells meaning that the transfection reaction was supplemented with 14 μg UltraPure Herring Sperm carrier DNA (ThermoFisher). 72 hours after the transfection, fresh baculovirus expressing AAV2 Replicase (Bac.VD183) was inoculated into the cells at 1% of the final volume (40 ml). 72 hours after the baculovirus infection the AAV batches were harvested by adding 10× lysis buffer (1.5 M NaCl, 0.5 M Tris-HCl, 1 mM MgCl₂, 10% Triton X-100, pH=8.5) and lysing for 1 hour at 28° C. Genomic DNA was digested by Benzonase treatment for 1 hour at 37° C. Cell debris was removed by centrifugation for 15 minutes at 1900×g after which the supernatant containing the AAV2/5 or AAV9 library was stored at 4° C. AAV2/5 or AAV9 library particles were purified from crude lysates with a batch binding protocol using AVB Sepharose (AAV2/5 library) affinity resin (GE Healthcare) or AAVx affinity resin (AAV9, ThermoFisher). In brief, AAV crude cell lysates were added to washed (with 0.2 M HPO₄ pH=7.5 buffer) resin. Subsequently, samples were incubated for 2 hours at room temperature under gentle mixing. Following the binding the resin was washed in 0.2 M HPO₄ PH=7.5 buffer and bound vectors were eluted by the addition of 0.2 M Glycine pH=2.5. The pH of the eluted vectors was immediately neutralized by the addition of 0.5 M Tris-HCl PH=8.5. Purified AAV batches were stored at −20° C.

### 2.1.3 Analysis of AAV2/5 and AAV9 Library Batches by QPCR and SDS PAGe Gel Electrophoresis

Titers of lysed or purified AAV2/5 or AAV9 batches were determined with a Q-PCR specific for the hr4b enhancer. AAVs were treated with DNAse at 37° C. to degrade exogenous DNA. AAV DNA was then released from the particles by a short (30 min) heat treatment (37° C.) in the presence of 1 M NaOH. Next the alkaline environment was neutralized by the addition of an equal volume of 1 M HCl. Neutralized DNA was diluted 10× in WFI 16 ng/μl PolyA, after which the samples were used in a QPCR with primers specific for the hr4b element on the transgene; forward CGAGGGATGATGTCATTTGTAGA (SEQ ID NO. 17), reverse ATCCACCGATCTTGCGTTAC (SEQ ID NO. 18) and probe Fam-ACCGAACTCGCTTTACGAGTAGAATTCT-mgb (SEQ ID NO. 19). VP protein composition of purified AAV2/5 or AAV9 libraries were determined by SDS page gel electrophoresis. Briefly, 15 μl of purified AAV2/5 was mixed with 5 μl 4× Leammli loading buffer (Biorad) supplemented with B-mercaptoethanol (Biorad). Following a short heat treatment to denature the proteins (5 min at 95° C.) material was loaded on a Stain free polyacrylamide gel (Biorad). Next, the samples were electrophoretic ally separated for 35 minutes at 200 Volts. Following electrophoresis stain (tryptophan based) was developed under UV-light for 5 minutes after which VP proteins were then visualized under UV light on a Chemidoc imaging system (Biorad).

### 2.1.4 Sanger and Whole Genome Sequencing of AAV2/5 and AAV9 Libraries

Viral transgene DNA was isolated from the AAV2/5 and AAV9 libraries using the Purelink viral DNA/RNA mini kit (ThermoFisher) following the kits protocol. A fragment containing the library insertion was amplified using PCR with primers specific for the VR-VIII insertion site (AAV2/5 VR-VIII fwd ACCAGCGAGAGCGAGAC and rev CTGCCGGGCACGATTTC, AAV9 VR-VIII fwd AAAGCCTGGACCGACTAATG and reverse TCTGTCCTGCCAAACCATAC; SEQ ID Nos. 20-23, respectively). Following column purification of the PCR product (Machery-Nagel), presence of the randomized insertion was verified by Sanger sequencing (using the same primers as for the PCR amplification, Macrogen) and Next generation sequencing (Genomscan). Whole genome sequencing was performed with the following setting; a Paired end 150 bp sequencing run was performed on the NovaSeq 6000 (illumina), 3 Gb of data was acquired per library. DNA was aligned using the barcode detection algorithm https://github.com/Jonas Weinmann/AAV-barcode-detection-and-normalization.

### 2.1 Results

This Example investigates if the AAV2/5 (pIMRaku-04) and AAV9 (plMRaku-11) scaffolds can be used for the production of AAV libraries in which randomized 7mer peptides are inserted at variable loop VR-VIII. Furthermore we investigate maintenance of complexity introduced into AAV5 and AAV9 capsid genes with Gibson assembly through various process steps of AAV library generation.

Randomized 21 bp DNA libraries were inserted at variable loop VR-VIII of the AAV2/5 and AAV9 capsid genes using the GRIT process previously described in FIG. 3. The AAV2/5 and AAV9 libraries were produced using the transfection infection method described in Example 1. The DNA scaffolds used in this Example are summarized in FIG. 7 and contain the same elements as previously described in FIG. 4b, with the only difference the capsid expression cassette being either AAV2/5 (pIMRAku04) or AAV9 (pIMRaku11). To investigate the producibility of the library, both the AAV2/5 and AAV9 DNA libraries scaffolds were transfected at multiple concentrations into the insect cells. Following transfection with library DNA scaffold and infection with AAV2 Rep baculovirus (SEQ ID NO. 14), crude cell lysates where harvested. AAV libraries where purified from lysates with AVB Sepharose (AAV2/5 library) or AAVx POROS (AAV9 library) affinity resins.

A QPCR specific for the hr4b enhancer was used to determine the viral titers (in gc/ml) of the crude lysate (CLB, AAV2/5 libraries) or purified (BBNE, AAV9 libraries) libraries (FIG. 8). Both for the AAV2/5 and AAV9 libraries we observed reduced producibility versus the plasmid controls without library insert. The drop in producibility of the AAV2/5 library is approximately 2 logs versus the plasmid control and 1 log versus the clean scaffold productions of Example 1 (Table 1). For the AAV9 library the drop in producibility seems to be roughly 1,5 log versus the plasmid control. Since not all inserts introduced in the AAV2/5 or AAV9 capsids DNA libraries will be able to produce capsids (due to misfolding of the capsid) a reduced titer was therefore expected for the AAV libraries. In addition, we observe a reduced production of the AAV library when less library DNA is transfected into the insect cells. Overall introducing complexity into the scaffold reduces the productivity for both serotypes. However the titers of the libraries remain sufficiently high to be useable for in vivo directed evolution studies. Production conditions for libraries to be used in directed evolution studies will have to be balanced based on the AAV library titer needs for the in vivo experiment and the need to reduce cross packaging.

Purified AAV2/5 and AAV9 libraries where also resolved by SDS PAGE to determine capsid stoichiometry and VP size (FIG. 8). Capsid stoichiometry falls in the natural VP123 ratio of approximately 1:1:10 for AAV for both the AAV2/5 and AAV9 libraries. Interestingly, the VP proteins of both the AAV2/5 and AAV9 appear to be slightly heavier than the plasmid controls (as seen by the slight upshift in fragment size of the libraries on the gel, noted by the arrows in FIG. 8). This can be explained by the 7aa insertions that were made in VP proteins of the libraries. This should result in a heavier capsid and hence the shift of the VP proteins of the library on the gel.

Next, we investigated if the insertion of the randomized 21 bp fragment into our DNA scaffold was successfully introduced and transferred during the AAV library production process. A primer set specific for the VR-VIII regions of the AAV2/5 and AAV9 capsid genes was used to amplify the region surrounding the library insertion site by PCR. This PCR was performed on intermediate samples taken while producing the AAV2/5 and AAV9 libraries. The intermediates included 1. the original library G-Block (AA2/5), 2. Gibson assembly (AA2/5), 3. RCA library amplification reaction (AAV2/5), 4, Un-purified lysate of the library AAV production (CLB, AAV2/5 and AAV9) and 5. Purified library (BBNE, AAV2/5 and AAV9). The amplified PCR product was analyzed for the presence of the library insertion via Sanger sequencing. This analysis gives a qualitative assessment on the presence of the library in the intermediate as the randomized DNA insertion should yield mixed reads in the sequence chromatogram at the insertion site. Sequence analysis confirmed presence of mixed reads at the insertion site throughout the production process for both the AAV2/5 and AAV9 libraries (FIG. 9). This result verifies that that our library amplification (GRIT) and AAV production (transfection/infection) method allows us to introduce and maintain the insertion throughout the AAV library production-process.

In the final assessment of the AAV2/5 library we monitored the complexity of the 21 bp insertion and quantitatively tracked its maintenance throughout the AAV library production process. On the PCR product previously generated for Sanger sequencing we performed next generation sequencing analysis. We acquired 3 GB of 150 bp paired end reads on a NovaSeq 6000 system on the following intermediate samples: 1) the original library G-Block; 2) the RCA library amplification reaction; and, 3) the purified library. We then aligned the obtained sequence reads against the AAV2/5 capsid cassette and quantified the number of the insertions found in the data pool using the barcode detection algorithm https://github.com/Jonas Weinmann/AAV-barcode-detection-and-normalization. We found that of the 1.22×10⁷ 21 bp insertion variants detected in the starter G-Block, 8.1×10⁶ carried over to the amplified DNA library of which 3.2×10⁶ variants were ultimately transferred over to the AAV2/5 capsid library (FIG. 10). Over the entire process 25% of the original complexity present in the G-Block gets transferred to the AAV capsid library, with the majority of diversity lost during the AAV production. This loss is likely due to the high percentage of misfolded AAV capsids due to unviable insertions. However, complexity is well maintained during the library DNA amplification process likely due to the rolling circle amplification method used for the scaffold generation.

In summary, we developed a DNA scaffold that is suitable for the production of AAV via a transfection/baculovirus infection based process in insect cells. The GRIT process is capable of inserting random 21 bp DNA insertions into this scaffold and amplifying them without a significant loss of complexity. AAV productions with these DNA libraries result in AAV libraries of sufficient concentration, and complexity to make them useable in directed evolution studies.

### 3. Example 3: Comparative Analysis of Genome Cross-Packaging in AAV Libraries Produced in Hek293t and expresSF+ Insect Cells

### 3.1 Methods

### 3.1.1 AAV9f and AAV2/5 DNA Scaffold Generation and AAV Production in HEK293t and Insect Cells

Linear DNA suitable for AAV9F and AAV2/5 capsid production in either Hek293t or expresSF+ was generated from plasmids containing capsid expression cassettes for AAV9F or AAV2/5 (HEK293t AAV2/5 (SEQ ID NO. 63) and HEK293t AAV9F (SEQ ID NO. 64) and SF+ AAV2/5 (SEQ ID NO. 65) and SF+ AAV9f (SEQ ID NO. 66). AAV9F and AAV2/5 DNA scaffold was generated by RCA followed by TELn digestion using the method described in example 1. In expresSF+ insect cells AAV was produced by transfecting 1.5e7 cells with 1 μg of plasmid using cellfectin II transfection reagent (ThermoFisher). 1 ug of a 1:1 mixture of scaffold pVD1943 (AAV2/5) and pVD1950 (AAV9F) was transfected into SF+. The transfection was performed according to the method described in example 1. In HEK293t cells, AAV was produced by transfecting 1e7 adherent HEK293t cells with 1 μg of plasmid using PEI (Polysciences). 1 ug of a 1:1 mixture of scaffold pVD2004 (AAV2/5) and pVD2006 (AAV9F) was transfected into HEK293t. 15 μg of total DNA was transfected into the cells meaning that the transfection reaction was supplemented with 14 μg UltraPure Herring Sperm carrier DNA (ThermoFisher). Transfections for both cell lines were performed in triplicate. 144 hours after transfection of the expresSF+ AAV and 72 hours after the transfection of the Hek293t AAV was harvested with the protocol described in example 1.

3.1.2 Sequential Purification of AAV2/5 and AAV9f Particles by Affinity Batch Binding with Serotype Specific Resins

AAV2/5 and AAV9f mixtures produced with expresSF+ or HEK293t were subjected to sequential batch binding purification. This sequential purification used affinity resins specific for either AAV5 (AVB Sepharose, Cytiva) or AAV9 particles (Poros9, Thermofisher). Crude lysate from the two capsid library productions was used first purified with AVB Sepharose resin (binding the AAV2/5 capsids), after which the flowthrough from this purification (containing the unbound AAV9 capsids) was used for a purification with Poros9 resin (binding AAV9 capsids). In brief the purification protocol: AAV crude cell lysates were added to washed (with 0.2M HPO₄ PH=7.5 buffer) resin (AVB sepharose or Poros9). Subsequently, samples were incubated for 2 hours at room temperature under gentle mixing. Following the binding the resin was washed in 0.2M HPO₄ PH=7.5 buffer and bound vectors were eluted by the addition of 0.2M Glycine pH=2.5. The pH of the eluted vectors was immediately neutralized by the addition of 0.5M Tris-HCl PH=8.5. Purified AAV batches were stored at −20° C.

3.1.3 Genome Identification by QPCR with Serotype Specific Primers

The identity of the genomes packaged in the sequentially purified particles produced with expresSF+ or HEK293t cells was analyzed with a QPCR specific for the AAV serotype. DNA was isolated from the AAV particles with the method described in example 1. The primers used for this QPCR are summarized in table 3 (SEQ ID NO. 55 to 60).

TABLE 3
Q-PCR primer sets used for serotype specific genome identification

| QPCR genome | | |
|---|---|---|
| target | Primer | Sequence |
| AAV2/5 genome | Primer fwd | CCCAGAGATCCAGTACACAAAC |
| specific QPCR | Primer rev | TCGGGTAAGGTATCGGGTT |
| | Probe | FAM-CGATAGGTCTGGTGGTTCTGTATTCCC |
| AAV9f genome | Primer fwd | TGATGGGAGGGTTTGGAATG |
| specific QPCR | Primer rev | AGAGTTCAGCTTGTCCTTGTT |
| | Probe | FAM-AAACACACCTGTACCTGCGGATCC |

### 3.1.4 Genome Identification by Next Generation Sequencing

The identity of genomes packaged in the sequentially purified particles produced with expresSF+ was also analyzed with next generation sequencing. Viral genomic DNA was isolated from the sequentially purified particles with the Purelink Viral RNA/DNA mini kit. Next the region of the genome that encodes the capsid was amplified by PCR using the following primers: forward CCTGCTGTTCCGAGTAACCATC and reverse GTCGGCGTGGTTGTACTTGAGG (SEQ ID NO. 61 and 62). The same primer set was used to amplify AAV2/5 and AAV9F genomes. PCR samples were send for illumina sequencing with a depth of 10 million reads. Genome identity was analyzed with a barcode counting script obtained from github: https://github.com/JonasWeinmann/AAV-barcode-detection-and-normalization.

### 3.1 Results

Cross-packaging occurs during production of AAV libraries when a genome that encodes for a capsid X is packaged into a mismatched capsid Y and takes places during library production (FIG. 11). In this example we investigate the rate of cross-packaging of our novel library production method in insect cells and compared it against standard library production method in HEK293t. We produced a library that contains a mixture two serotypes (AAV2/5 and AAV9F) using either the insect cell or HEK293t production methods. Following sequential purification with affinity resins that selectively purify AAV2/5 or AAV9F capsids we used serotype specific QPCRs to identify the origin of the genomes packaged inside these AAV capsids. By determining the fraction of mismatched genomes in the pools sampled after serotype selective purification (for example AAV9F genomes in AAV2/5 capsids) we can establish the % cross-packaging of the library production method (FIG. 12A). This simplified experiment of assessing cross-packaging in our library is based on a method previously described to assess cross-packaging in HEK293t cells developed by Nonnenmacher at all 2015.

FIG. 12B shows the % of cross-packaging as measured by QPCR in AAV particle pools obtained from a two capsid library after sequential serotype specific purification. A significant reduction in % cross-packaging was observed in the two capsid library produced in expresSF+ cells when compared to the two capsid library produced in HEK293t cells. This was the case for both AAV9F genomes found in AAV2/5 capsids and AAV2/5 genomes found in AAV9F capsids. NGS analysis of % of cross-packaging on the samples produced in expresSF+ cells revealed similar levels of cross-packaging as we measured with the serotype specific QPCR (table 4).

In summary, we measured significantly reduced cross-packaging rates in the two capsid library produced with our method in insect cells versus the industry standard in HEK293t cells. While this method only gives a simplified view on the cross-packaging process, similar methods have been used to establish cross-packaging for AAV libraries produced in HEK293t cells. Low levels of cross-packaging are correlated with an increased speed of directed evolutionary studies aimed at selecting capsids with novel properties. By having reduced cross-packaging with our capsid library production method in insect cells we anticipate faster selection of novel in upcoming studies.

TABLE 4
NGS analysis of crosspackaging in two capsid library
produced in expresSF + insect cells. Similar
levels in cross-packaging were measured in insect
cell produced libraries with both NGS and QPCR.

| | % cross-packaging | % cross-packaging |
|---|---|---|
| | by NGS | by QPCR |
| Sample 1 SF+: AAV9f | 2.29 | 2.22 |
| genomes in AAV2/5 | | |
| capsids | | |
| Sample 2 SF+: AAV2/5 | 2.62 | 3.52 |
| genomes in AAV9f | | |
| capsids | | |

1. A linear nucleic acid molecule comprising a nucleic acid construct comprising:
i) a nucleotide sequence encoding an adeno-associated virus (AAV) capsid variant, wherein the nucleotide sequence encodes an mRNA, translation of which in an insect cell produces the AAV capsid variant, and wherein the nucleotide sequence is operably linked to a promoter for driving expression in an insect cell;
ii) an enhancer element that is operably linked to the promoter, wherein the enhancer element is dependent on a transcriptional transregulator, and wherein introduction of the transcriptional transregulator into an insect cell comprising the nucleic acid construct, induces transcription from the promoter;
iii) at least one AAV inverted terminal repeat (ITR) sequence;
iv) optionally, a reporter gene operably linked to a promoter for driving expression in a mammalian cell; and,
v) optionally, a barcode.

2. The nucleic acid molecule according to claim 1, the transcriptional transregulator is a baculoviral immediate-early protein (IE1) or its splice variant (IE0) and the transcriptional transregulator-dependent enhancer element is a baculoviral homologous region (hr) enhancer element, wherein preferably the baculovirus is Autographa californica multicapsid nucleopolyhedrovirus.

3. The nucleic acid molecule according to claim 2, wherein the hr enhancer element comprises at least one copy of the hr 28-mer sequence CTTTACGAGTAGAATTCTACGCGTAAAA and/or at least one copy of a of a sequence of which at least 20, 21, 22, 23, 24, 25, 26, or 27 nucleotides are identical to sequence CTTTACGAGTAGAATTCTACGCGTAAAA and which binds to a baculoviral IE1 protein, and wherein the hr enhancer element, when operably linked to an expression cassette comprising a reporter gene operably linked to the polH promoter,
a) under non-inducing conditions, the expression cassette with the hr enhancer element produces less reporter transcript than an otherwise identical expression cassette which comprises the hr2-0.9 element, or the cassette with the hr enhancer element produces less than a factor 1.1, 1.2, 1.5, 2, 5 or 10 of the amount reporter transcript produced by an otherwise identical expression cassette which comprises the hr4b element; and,
b) under inducing conditions, the expression cassette with the hr enhancer element produces at least 50, 60, 70, 80, 90 or 100% of the amount of reporter transcript produced by an otherwise identical expression cassette which comprises the hr4b or the hr2-0.9 element,
wherein more preferably the hr enhancer element is selected from the group consisting of hr1, hr2-0.9, hr3, hr4b and hr5, of which hr4b and hr5 are preferred, of which hr4b is most preferred.

4. The nucleic acid molecule according to claim 1, wherein the promoter for driving expression in an insect cell is a baculoviral promoter, preferably, a late or very late baculoviral promoter, more preferably, a promoter selected from the group consisting of the polH, p10, p6.9 and pSel120 promoters.

5. The nucleic acid molecule according to claim 1, wherein the linear nucleic acid molecule is obtainable by digestion with a site specific endonuclease that cuts at a recognition sequence in the nucleic acid construct.

6. The nucleic acid molecule according to claim 1, wherein at least one end of the linear nucleic acid molecule is protected against exonuclease degradation, preferably both ends of the linear nucleic acid molecule are protected against exonuclease degradation.

7. The nucleic acid molecule according to claim 5, wherein the endonuclease is a protelomerase, preferably a phage N15 TelN protelomerase, and wherein at least one end of the linear nucleic acid molecule is covalently closed, or wherein preferably both ends of the linear nucleic acid molecule are covalently closed.

8. The nucleic acid molecule according to claim 5, wherein the locations of elements i) to v) and the recognition sequence in the construct are such that expression of AAV Rep proteins in an insect cell comprising the nucleic acid construct, effects replication and packaging into AAV capsids of a DNA fragment comprising elements i) to v).

9. The nucleic acid molecule according to claim 1, wherein the nucleic acid construct comprises in a 5′ to 3′ order:
a) a first AAV ITR sequence;
b) the enhancer element;
c) an expression cassette comprising the nucleotide sequence encoding the AAV capsid variant, operably linked to the promoter for driving expression in the insect cell; and,
d) a second AAV ITR,
wherein the recognition sequence is present at at least one of i) upstream of the first AAV ITR and ii) downstream of the second AAV ITR,
wherein optionally, the reporter gene operably linked to the promoter for driving expression in a mammalian cell is present between a) and b), b) and c) or c) and d), and wherein optionally, a barcode is present between a) and b), b) and c) or c) and d) and wherein, preferably the recognition sequence is a unique recognition sequence.

10. The nucleic acid molecule according to claim 1, wherein at least one of:
a) the promoter for driving expression in a mammalian cell is a cell type and/or tissue specific promoter, preferably a promoter selected from the group consisting of human synapsin promoter (hSynl), transthyretin promoter (TTR), cytokeratin 18, cytokeratin 19, unc-45 myosin chaperon B (unc45b) promoter, cardiac troponin T (cTnT) promoter, glial fibrillary acidic protein (GFAP) promoter, myelin basic protein (MBP) promoter, and methyl-CpG-binding protein 2 (Mecp2) promoter;
b) the reporter gene encodes a colorimetric, fluorescent or bioluminescent reporter protein, preferably a reporter protein is selected from the group consisting of: secreted alkaline phosphatase (SEAP), β-galactosidase EGFP, mCherry, mCloverS, mRubyS, mApple, iRFP, tdTomato, m Venus, YFP, RFP, firefly luciferase, sea pansy luciferase and nanoluciferase; and,
c) the barcode is a nucleotide sequence of 4-18 nucleotides long.

11. A library comprising a plurality of nucleic acid molecules comprising a construct as defined in claim 1, wherein, in each member of the library, the nucleotide sequence encoding the AAV capsid variant differs by at least one nucleotide from nucleotide sequences encoding the AAV capsid variants in other members in the library, and wherein preferably, in each member of the library, the nucleotide sequence encoding the AAV capsid variant, encodes a capsid variant that differs by at least one amino acid from AAV capsid variants encoded by other members in the library.

12. An insect cell comprising a nucleic acid molecule comprising a construct as defined in claim 1.

13. The insect cell according to claim 12, further comprising a baculoviral vector comprising at least one expression cassette for expression of AAV Rep proteins in the insect cell.

14. (canceled)

15. A method for preparing a library of AAV capsid variants, the method comprising the steps of:
a) providing a library comprising a plurality of nucleic acid construct as defined claim 1, wherein the amino acid sequence of an AAV capsid variant of each member in the library as encoded in the construct of that member, differs by at least one amino acid from amino acid sequences of AAV capsid variants encoded in constructs of other members in the library;
b) amplifying the library of the nucleic acid constructs obtained in a), preferably by rolling circle amplification;
c) digestion of the amplified library of the nucleic acid constructs obtained in b) by the endonuclease to produce a library comprising linearised amplified nucleic acid constructs of unit length;
d) transfection of the library comprising linearised amplified nucleic acid constructs obtained in c) into insect cells;
e) infection of the insect cells with a baculoviral vector comprising at least one expression cassette for expression of AAV Rep proteins in the insect cells and culturing of the cells; and,
f) recovery of a library of AAV capsid variants produced by the insect cells in e).

16. The method according to claim 15, wherein the library in a) is provided by:
i) providing a nucleic acid construct as defined in claim 1; and,
ii) replacing a fragment comprising at least a part of the nucleotide sequence encoding the AAV capsid variant in the construct, by a library of fragments, wherein the amino acid sequence of an AAV capsid variant of each member in the library as encoded in the fragment of that member, differs by at least one amino acid from amino acid sequences of AAV capsid variants encoded in constructs of other members in the library.

17. A The method according to claim 16, wherein:
in step ii), the replacement is performed by Gibson assembly.

18. The method according to claim 15, wherein:
in step b), the rolling circle amplification is performed by using Φ29 DNA polymerase, preferably, in combination with random primers; and/or,
in c), the ends of the linearised amplified library of the nucleic acid constructs of unit length are provided with protection against exonuclease degradation of wherein
the endonuclease is a phage N15 TelN protelomerase, providing covalently closed ends to the linearised amplified library of the nucleic acid constructs of unit length.

19. A method for identifying an AAV capsid variant with a desired characteristic, the method comprising:
a) providing a library of AAV capsid variants wherein members in the library comprise a nucleic acid that is replicated and packaged from a member in the library of nucleic acid constructs, or as obtained in a method according to claim 15;
b) contacting the library with a culture of cells, an organoid or a tissue, or administering the library to a non-human animal;
c) allowing AAV capsid variants in the library to transduce the cells, the organoid, the tissue or cells in the animal; and,
d) identifying at least one AAV capsid variant that has transduced at least one desired cell-type in the cells, the organoid, the tissue or in the animal in b), as an AAV capsid variant with the desired characteristic, and optionally recovery of the AAV capsid variant with the desired characteristic from the desired cell.

20. The method according to claim 19, wherein step d) comprises detection of transduction of at least the desired cell by detecting expression of the reporter gene in the desired cell.

21. The method according to claim 19, wherein the variant with the desired characteristic is identified by sequencing at least one of i) the barcode and ii) a region of the DNA construct encoding the at least one amino acid difference.


SEQUENCE LISTING (66 sequences)

SEQ ID NO: 1 - DNA, 28 bp, synthetic construct
  source 1..28: mol_type = other DNA; note = hr 28-mer
ctttacgagtagaattctacgcgtaaaa

SEQ ID NO: 2 - DNA, 56 bp, synthetic construct
  source 1..56: mol_type = other DNA; note = TeIN protelomerase
tatcagcacacaattgcccattatacgcgcgtataatggactattgtgtgctgata

SEQ ID NO: 3 - DNA, 714 bp, synthetic construct
  source 1..714: mol_type = other DNA; note = Rep52 wt
aaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 4 - DNA, 720 bp, synthetic construct
  source 1..720: mol_type = other DNA; note = rep52 sf9 (insect cell) optimised
atggagctggtgggttggctggtggacaagggtatcacctccgagaagcagtggatccaggaggaccaggcttcctacatctccttcaacgctgcttccaactcccgttcccagatcaaggctgctctggacaacgctggtaagatcatgtccctgaccaagaccgctcctgactacctggtgggtcagcagcctgtggaggacatctcctccaaccgtatctacaagatcctggagctgaacggttacgaccctcagtacgctgcttccgtgttcctgggttgggctaccaagaagttcggtaagcgtaacaccatctggctgttcggtcctgctaccaccggtaagaccaacatcgctgaggctatcgctcacaccgtgcctttctacggttgcgtgaactggaccaacgagaacttccctttcaacgactgcgtggacaagatggtgatctggtgggaggagggtaagatgaccgctaaggtggtggagtccgctaaggctatcctgggtggttccaaggtgcgtgtggaccagaagtgcaagtcctccgctcagatcgaccctacccctgtgatcgtgacctccaacaccaacatgtgcgctgtgatcgacggtaactccaccaccttcgagcaccagcagcctctgcaggaccgtatgttcaagttcgagctgacccgtcgtctggaccacgacttcggtaaggtgaccaagcag

SEQ ID NO: 5 - DNA, 714 bp, synthetic construct
  source 1..714: mol_type = other DNA; note = rep52 AT optimised
aaagtagtagaatcagcaaaagcaatattaggaggatcaaaagtaagagtagatcaaaaatgtaaatcatcagcacaaatagatccaacaccagtaatagtaacatcaaatacaaatatgtgtgcagtaatagatggaaattcaacaacatttgaacatcaacaaccattacaagatagaatgtttaaatttgaattaacaagaagattagatcatgattttggaaaagtaacaaaacaagaagtaaaagatttttttagatgggcaaaagatcatgtagtagaagtagaacatgaattttatgtaaaaaaaggaggagcaaaaaaaagaccagcaccatcagatgcagatatatcagaaccaaaaagagtaagagaatcagtagcacaaccatcaacatcagatgcagaagcatcaataaattatgcagatagatatcaaaataaatgttcaagacatgtaggaatgaatttaatgttatttccatgtagacaatgtgaaagaatgaatcaaaattcaaatatatgttttacacatggacaaaaagattgtttagaatgttttccagtatcagaatcacaaccagtatcagtagtaaaaaaagcatatcaaaaattatgttatatacatcatataatgggaaaagtaccagatgcatgtacagcatgtgatttagtaaatgtagatttagatgattgtatatttgaacaataa

SEQ ID NO: 6 - DNA, 714 bp, synthetic construct
  source 1..714: mol_type = other DNA; note = rep52 GC optimised
aaggtggtggagagcgcgaaggcgatcctgggggggagcaaggtgcgggtggaccagaagtgcaagagcagcgcgcagatcgacccgacgccggtgatcgtgacgagcaacacgaacatgtgcgcggtgatcgacgggaacagcacgacgttcgagcaccagcagccgctgcaggaccggatgttcaagttcgagctgacgcggcggctggaccacgacttcgggaaggtgacgaagcaggaggtgaaggacttcttccggtgggcgaaggaccacgtggtggaggtggagcacgagttctacgtgaagaaggggggggcgaagaagcggccggcgccgagcgacgcggacatcagcgagccgaagcgggtgcgggagagcgtggcgcagccgagcacgagcgacgcggaggcgagcatcaactacgcggaccggtaccagaacaagtgcagccggcacgtggggatgaacctgatgctgttcccgtgccggcagtgcgagcggatgaaccagaacagcaacatctgcttcacgcacgggcagaaggactgcctggagtgcttcccggtgagcgagagccagccggtgagcgtggtgaagaaggcgtaccagaagctgtgctacatccaccacatcatggggaaggtgccggacgcgtgcacggcgtgcgacctggtgaacgtggacctggacgactgcatcttcgagcagtaa

SEQ ID NO: 7 - DNA, 714 bp, synthetic construct
  source 1..714: mol_type = other DNA; note = Extreme codon optimized Rep52
aaagtggttgaatccgcgaaggcaatcctgggtggttccaaagtccgtgtcgatcaaaagtgtaaaagctccgctcaaatcgatcctaccccggtaattgttacgagtaatactaatatgtgcgcagttattgacggtaactcgacgacgtttgaacatcaacaacccctccaggatagaatgtttaagtttgagctgactagacgcctcgaccacgatttcggtaaagttactaaacaggaggtgaaggatttctttcgctgggctaaagaccatgtcgtagaagtcgagcacgagttttatgttaagaaaggaggtgctaaaaagcgtcctgctcctagcgatgctgacatctccgaacctaagcgtgtccgtgaaagcgtcgcacaacctagcaccagcgatgctgaggcctccattaattatgctgatcgttatcagaataagtgcagccgccatgtcggtatgaacctcatgctcttcccttgtcgtcagtgtgaacgtatgaaccaaaactcaaacatatgttttacccatggtcaaaaggactgcctggagtgcttccctgtaagtgaatcgcaacctgtaagtgtggtaaagaaagcgtaccaaaagctctgttatatccaccacattatgggcaaagtaccggatgcctgtaccgcgtgtgaccttgtgaacgttgacctggacgattgtatattcgagcagtaa

SEQ ID NO: 8 - DNA, 673 bp, synthetic construct
  source 1..673: mol_type = other DNA; note = Rep78
caaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 9 - DNA, 4423 bp, synthetic construct
  source 1..4423: mol_type = other DNA; note = pIMRAku-04 AAV2/5 DNA scaffold
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacggcgcgccttgacgcaaatgggcggtaggcgtgtacggtgggaggtctatataagcagagctcgtttagtgaaccgtcagatcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggctagcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtgctggagcaaggaggtgtgtccagtttgtttcagaatctcggggtgtccgtaactccgatccaaaggattgtcctgtccggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgaaggagtggacggacctttaattcaacccaacacaatatattatagttaaataagaattattatcaaatcatttgtatattaattaaaatactatactgtaaattacattttatttacaatcaatttaaatacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagatgagaagaccacctgcgatctcgccgttcagtctcgtccaagggcacgcgctcgcgaataaaagatctttattttcattagatctgtgtgttggttttttgtgtgatgcagttaacatcgataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggccggcctatcagcacacaattgcccattatacgcgcgtataatggactattgtgtgctgata

SEQ ID NO: 10 - DNA, 2416 bp, synthetic construct
  source 1..2416: mol_type = other DNA; note = AAV2/5 expression cassette
acggacctttaattcaacccaacacaatatattatagttaaataagaattattatcaaatcatttgtatattaattaaaatactatactgtaaattacattttatttacaatcaatttaaatacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagatgagaagaccacctgcgatctcgccgttcagtctcgtccaagggcacgcgctcgcgaataaaagatctttattttcattagatctgtgtgttggttttttgtgtgatgca

SEQ ID NO: 11 - DNA, 4477 bp, synthetic construct
  source 1..4477: mol_type = other DNA; note = pIMRAku-011 AAV9 Scaffold
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacggcgcgccttgacgcaaatgggcggtaggcgtgtacggtgggaggtctatataagcagagctcgtttagtgaaccgtcagatcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggctagcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtgctggagcaaggaggtgtgtccagtttgtttcagaatctcggggtgtccgtaactccgatccaaaggattgtcctgtccggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgaaggagtggacggacctttaattcaacccaacacaatatattatagttaaataagaattattatcaaatcatttgtatattaattaaaatactatactgtaaattacattttatttacaatcaatttaaatacggctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtcaggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatttgtagtaggacagagctatgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatctagatgagaagaccacctgcgatctcgccgttcagtctcgtccaagggcacgcgctcgcgaataaaagatctttattttcattagatctgtgtgttggttttttgtgtgatgcagttaacatcgataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggccggcctatcagcacacaattgcccattatacgcgcgtataatggactattgtgtgctgata

SEQ ID NO: 12 - DNA, 2534 bp, synthetic construct
  source 1..2534: mol_type = other DNA; note = AAV9 expression cassette
tacggacctttaattcaacccaacacaatatattatagttaaataagaattattatcaaatcatttgtatattaattaaaatactatactgtaaattacattttatttacaatcactcgacgaagacttgatcacccctagattcgaaagcggcccgctcactagtaacggccgccagtgtgctggaattcgcccttcgcggatcctgttaagacggctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctgcacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcagaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatgagaagaccacctgcgatctcgccgttcagtctcgtccaagggcgtcgactcgcgaataaaagatctttattttcattagatctgtgtgttggttttttgtgtgatgca

SEQ ID NO: 13 - DNA, 4123 bp, synthetic construct
  source 1..4123: mol_type = other DNA; note = AAV2 Replicase expression cassette
gtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 14 - DNA, 529 bp, synthetic construct
  source 1..529: mol_type = other DNA; note = AAV2/5 c7c library gblock
  misc_difference 351..364: note = n may be any nucleotide.
cctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctgcnnnnnnnnnnnnnntgtactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacac

SEQ ID NO: 15 - DNA, 432 bp, synthetic construct
  source 1..432: mol_type = other DNA; note = AAV9 7mer library gblock
  misc_difference 185..198: note = n may be any nucleotide.
caaagaaggagaggaccgtttctttcctttgtcaggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaannnnnnnnnnnnnngcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaag

SEQ ID NO: 16 - DNA, 23 bp, synthetic construct
  source 1..23: mol_type = other DNA; note = hr4b fwd
cgagggatgatgtcatttgtaga

SEQ ID NO: 17 - DNA, 20 bp, synthetic construct
  source 1..20: mol_type = other DNA; note = hr4b rev
atccaccgatcttgcgttac

SEQ ID NO: 18 - DNA, 28 bp, synthetic construct
  source 1..28: mol_type = other DNA; note = hr4b probe
accgaactcgctttacgagtagaattct

SEQ ID NO: 19 - DNA, 17 bp, synthetic construct
  source 1..17: mol_type = other DNA; note = AAV2/5 VR-VIII fwd
accagcgagagcgagac

SEQ ID NO: 20 - DNA, 17 bp, synthetic construct
  source 1..17: mol_type = other DNA; note = AAV2/5 VR-VIII rev
ctgccgggcacgatttc

SEQ ID NO: 21 - DNA, 20 bp, synthetic construct
  source 1..20: mol_type = other DNA; note = AAV9 VR-VIII fwd
aaagcctggaccgactaatg

SEQ ID NO: 22 - DNA, 20 bp, synthetic construct
  source 1..20: mol_type = other DNA; note = AAV9 VR-VIII rev
tctgtcctgccaaaccatac

SEQ ID NO: 23 - DNA, 39 bp, synthetic construct
  source 1..39: mol_type = other DNA
gatggccaccaacaaccagagctccaccactgcccccgc

SEQ ID NO: 24 - DNA, 36 bp, synthetic construct
  source 1..36: mol_type = other DNA
ttttttgctttcttgcttggtttgtactgcccccgc

SEQ ID NO: 25 - DNA, 36 bp, synthetic construct
  source 1..36: mol_type = other DNA
ttgttggctgcctggcttggcttgtactgcccccgc

SEQ ID NO: 26 - DNA, 36 bp, synthetic construct
  source 1..36: mol_type = other DNA
gtttttggttggttggttggtttgtactgcccccgc

SEQ ID NO: 27 - DNA, 36 bp, synthetic construct
  source 1..36: mol_type = other DNA
cttttttttttttttttttttttgtactgcccccgc

SEQ ID NO: 28 - DNA, 36 bp, synthetic construct
  source 1..36: mol_type = other DNA
tgaatggctggctggctggctgtgtactgcccccgc

SEQ ID NO: 29 - DNA, 36 bp, synthetic construct
  source 1..36: mol_type = other DNA
tttgtttggttggttgttggtttgtactgcccccgc

SEQ ID NO: 30 - DNA, 31 bp, synthetic construct
  source 1..31: mol_type = other DNA
gtgcccaannknnknnknnknnknnknnkgc

SEQ ID NO: 31 - DNA, 31 bp, synthetic construct
  source 1..31: mol_type = other DNA
gtgcccaatttattaataataatattaatgc

SEQ ID NO: 32 - DNA, 31 bp, synthetic construct
  source 1..31: mol_type = other DNA
gtgcccaaaatactaataataatattaatgc

SEQ ID NO: 33 - protein, 48 aa, synthetic construct
ESETQPVNRVAYNVGGQMATNNQSCLSRRLYSCTAPATGTYNLQEIVP

SEQ ID NO: 34 - protein, 48 aa, synthetic construct
ESETQPVNRVAYNVGGQMATNNQSCHDHLVSRCTAPATGTYNLQEIVP

SEQ ID NO: 35 - protein, 48 aa, synthetic construct
ESETQPVNRVAYNVGGQMATNNQSCILKSAWWCTAPATGTYNLQEIVP

SEQ ID NO: 36 - protein, 48 aa, synthetic construct
ESETQPVNRVAYNVGGQMATNNQSCTRVRQSECTAPATGTYNLQEIVP

SEQ ID NO: 37 - protein, 48 aa, synthetic construct
ESETQPVNRVAYNVGGQMATNNQSCSQGPHFTCTAPATGTYNLQEIVP

SEQ ID NO: 38 - protein, 48 aa, synthetic construct
ESETQPVNRVAYNVGGQMATNNQSCIIVLFLVCTAPATGTYNLQEIVP

SEQ ID NO: 39 - protein, 48 aa, synthetic construct
ESETQPVNRVAYNVGGQMATNNQSCVACGCFICTAPATGTYNLQEIVP

SEQ ID NO: 40 - protein, 48 aa, synthetic construct
ESETQPVNRVAYNVGGQMATNNQSCDDFRPKSCTAPATGTYNLQEIVP

SEQ ID NO: 41 - protein, 48 aa, synthetic construct
ESETQPVNRVAYNVGGQMATNNQSCHLVCCPSCTAPATGTYNLQEIVP

SEQ ID NO: 42 - protein, 48 aa, synthetic construct
ESETQPVNRVAYNVGGQMATNNQSCHDMGRVSCTAPATGTYNLQEIVP

SEQ ID NO: 43 - protein, 48 aa, synthetic construct
ESETQPVNRVAYNVGGQMATNNQSCGFLPVFPCTAPATGTYNLQEIVP

SEQ ID NO: 44 - protein, 48 aa, synthetic construct
ESETQPVNRVAYNVGGQMATNNQSCLRFLYFMTCAPATGTYNLQEIVP

SEQ ID NO: 45 - protein, 48 aa, synthetic construct
ESETQPVNRVAYNVGGQMATNNQSCCLCPYIACTAPATGTYNLQEIVP

SEQ ID NO: 46 - protein, 48 aa, synthetic construct
ESETQPVNRVAYNVGGQMATNNQSCFQSHYYACTAPATGTYNLQEIVP

SEQ ID NO: 47 - protein, 48 aa, synthetic construct
ESETQPVNRVAYNVGGQMATNNQSCKPVLSTRCTAPATGTYNLQEIVP

SEQ ID NO: 48 - protein, 48 aa, synthetic construct
ESETQPVNRVAYNVGGQMATNNQSCVLEMPRNCTAPATGTYNLQEIVP

SEQ ID NO: 49 - protein, 48 aa, synthetic construct
ESETQPVNRVAYNVGGQMATNNQSCMFNWWHTCTAPATGTYNLQEIVP

SEQ ID NO: 50 - protein, 48 aa, synthetic construct
ESETQPVNRVAYNVGGQMATNNQSCYRVLMMVCTAPATGTYNLQEIVP

SEQ ID NO: 51 - protein, 48 aa, synthetic construct
ESETQPVNRVAYNVGGQMATNNQSCVTQAQTGCTAPATGTYNLQEIVP

SEQ ID NO: 52 - protein, 48 aa, synthetic construct
ESETQPVNRVAYNVGGQMATNNQSCFSHNLCICTAPATGTYNLQEIVP

SEQ ID NO: 53 - protein, 48 aa, synthetic construct
ESETQPVNRVAYNVGGQMATNNQSCXLVLYFSCTAPATGTYNLQEIVP

SEQ ID NO: 54 - protein, 48 aa, synthetic construct
ESETQPVNRVAYNVGGQMATNNQSCXXXXXXXCTAPATGTYNLQEIVP

SEQ ID NO: 55 - DNA, 22 bp, synthetic construct
  source 1..22: mol_type = other DNA
cccagagatccagtacacaaac

SEQ ID NO: 56 - DNA, 19 bp, synthetic construct
  source 1..19: mol_type = other DNA
tcgggtaaggtatcgggtt

SEQ ID NO: 57 - DNA, 27 bp, synthetic construct
  source 1..27: mol_type = other DNA
cgataggtctggtggttctgtattccc

SEQ ID NO: 58 - DNA, 20 bp, synthetic construct
  source 1..20: mol_type = other DNA
tgatgggagggtttggaatg

SEQ ID NO: 59 - DNA, 21 bp, synthetic construct
  source 1..21: mol_type = other DNA
agagttcagcttgtccttgtt

SEQ ID NO: 60 - DNA, 24 bp, synthetic construct
  source 1..24: mol_type = other DNA
aaacacacctgtacctgcggatcc

SEQ ID NO: 61 - DNA, 22 bp, synthetic construct
  source 1..22: mol_type = other DNA
cctgctgttccgagtaaccatc

SEQ ID NO: 62 - DNA, 22 bp, synthetic construct
  source 1..22: mol_type = other DNA
gtcggcgtggttgtacttgagg

SEQ ID NO: 63 - DNA, 6892 bp, synthetic construct
  source 1..6892: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacggcgcgccttgacgcaaatgggcggtaggcgtgtacggtgggaggtctatataagcagagctcgtttagtgaaccgtcagatcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggctagcaattatcgacgagcgcctgatcaaccccgacggctccctgctgttccgagtaaccatcaacggagtgaccggctggcggctgtgcgaacgcatttgtgaccagcatgcggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagatgagaagaccacctgcgatctcgccgttcagtctcgtccaagggcacgcgctcgcgaataaaagatctttattttcattagatctgtgtgttggttttttgtgtgatgcagttaacatcgataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggccggcctatcagcacacaattgcccattatacgcgcgtataatggactattgtgtgctgataggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccaacgcggcctttttacggttcctggccttttgctggccttttgctcacatgt

SEQ ID NO: 64 - DNA, 6946 bp, synthetic construct
  source 1..6946: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacggcgcgccttgacgcaaatgggcggtaggcgtgtacggtgggaggtctatataagcagagctcgtttagtgaaccgtcagatcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggctagcaattatcgacgagcgcctgatcaaccccgacggctccctgctgttccgagtaaccatcaacggagtgaccggctggcggctgtgcgaacgcattaggagtgggcatgcggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaagacaaccttagtgaaggaattcgcgagtggtgggctttgaaacctggagcccctcaacccaaggcaaatcaacaacatcaagacaacgctcgcggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtcaggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatttgtagtaggacagagctatgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatctagatgagaagaccacctgcgatctcgccgttcagtctcgtccaagggcacgcgctcgcgaataaaagatctttattttcattagatctgtgtgttggttttttgtgtgatgcagttaacatcgataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggccggcctatcagcacacaattgcccattatacgcgcgtataatggactattgtgtgctgataggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcctttttacggttcctggccttttgctggccttttgctcacatgt

SEQ ID NO: 65 - DNA, 6795 bp, synthetic construct
  source 1..6795: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacggcgcgccttgacgcaaatgggcggtaggcgtgtacggtgggaggtctatataagcagagctcgtttagtgaaccgtcagatctgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaagctagcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtgctggagcaaggaggtgtgtccagtttgtttcagaatctcggggtgtccgtaactccgatccaaaggattgtcctgtccggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgaacagtggtacggacctttaattcaacccaacacaatatattatagttaaataagaattattatcaaatcatttgtatattaattaaaatactatactgtaaattacattttatttacaatcaatttaaatacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagatgagaagaccacctgcgatctcgccgttcagtctcgtccaagggcacgcgctcgcgaataaaagatctttattttcattagatctgtgtgttggttttttgtgtgatgcagttaacatcgataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggccggcctatcagcacacaattgcccattatacgcgcgtataatggactattgtgtgctgataggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctcttttgctcacatgt

SEQ ID NO: 66 - DNA, 7071 bp, synthetic construct
  source 1..7071: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacggcgcgccttgacgcaaatgggcggtaggcgtgtacggtgggaggtctatataagcagagctcgtttagtgaaccgtcagatcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggctagcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtgctggagcaaggaggtgtgtccagtttgtttcagaatctcggggtgtccgtaactccgatccaaaggattgtcctgtccggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgaaggagtggacggacctttaattcaacccaacacaatatattatagttaaataagaattattatcaaatcatttgtatattaattaaaatactatactgtaaattacattttatttacaatcaatttaaatacggctgccgatggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtcaggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatttgtagtaggacagagctatgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatctagatgagaagaccacctgcgatctcgccgttcagtctcgtccaagggcacgcgctcgcgaataaaagatctttattttcattagatctgtgtgttggttttttgtgtgatgcagttaacatcgataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggccggcctatcagcacacaattgcccattatacgcgcgtataatggactattgtgtgctgataggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccaacgcggcctttttacggttcctggccttttgctggccttttgctcacatgt